SEQUENCE LISTING (29 sequences)

SEQ ID NO: 1 - DNA, 2913 bp, Zea mays
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggtgcagatcgcgcaccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccaggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcttctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggcccgaggacaagaggcaggagtggctgctgtcggagctgcgaggcaagcgcccgctgctgcccccggaccttccccagaccgacgagatcgccgacgtcatcggcgcgttccacgtcctcgcggagctcccgcccgacagcttcggcccctacatcatctccatggcgacggccccctcggacgtgctcgccgtggagctcctgcagcgcgagtgcggcgtgcgccagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctccgtcgtcgtcaaggaggcgcgcttcgtcgagtacttcagatcggctacaccggagaccgagtacgggaggatgaacatcggcagccggccagccaagaggaggcccggcggcggcatcacgaccctgcgcgccatcccctggatcttctcgtggacccagaccaggttccacctccccgtgtggctgggagtcggcgccgcattcaagttcgccatcgacaaggacgtcaggaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 2 - DNA, 2904 bp, Zea mays
atggctgccttagggccgaagatggagcgtctgtcgtcgatcgacgcgcagctgcggatgctggtgccggggaaggtttcggaggacgataagctcatcgagtacgacgctctcctcctcgatcggttcctcgacatccttcaggacctccatggcgacgacctcaaggaaatggttcaagaatgctatgaggtagctgctgagtatgaaacaaaacatgacttgcaaaagcttgatgaactcgggaagatgataacaagcttggatcctggggactctatcgtgattgctaagtctctctcgcacatgcttaacttggccaacttggctgaggaagtccagatagcctacaggaggagaatcaagctcaagaagggagactttgctgatgagaactcggcaatcacagaatctgacattgaggaaacacttaagaggcttgttgttgacctgaagaagtcacctgctgaggtatttgatgccctcaagagccagactgttgatctggttttgactgcacatccaacacagtctgtgaggaggtcactgctccagaaacactcgaggatacgcaactgtttggttcaactctactcaaaagatatcactccggatgataagcaggaacttgatgaggctctgcaaagagagatccaagctgcctttagaactgatgagatccgaagaacacagcccactccccaagatgaaatgcgtgctggtatgagctacttccacgaaacaatttggaagggtgttccaaagttcttgcgccgggttgatactgcattgaagaacattgggattaacgagcgtgttccttacaatgcacctcttattcaattctcttcttggatggggggagatcgtgatggaaatccaagagtgacaccagaggttaccagggatgtctgcttgcttgccagaatgatggcatcaaacttgtactgctctcagattgaggatctcatgtttgagttgtctatgtggcgatgcagtgatgaactgcgcatgcgagctgatgtgctgcatctctccactaagaaggatgccaaacattacatagagttttggaagaaggttcctccaaatgagccatatcgggtgatactgagtgatgtcagggataaactgtacaacactcgtgaacggtcacgagagcttttatccagtgggcattctgatattcctgaggaagctacattgacaaatgttgagcagctgttggagcccttggaactatgttacagatcactctgtgcttgtggtgactctgtaattgctgatggaacccttctggatttcttgcgtcaagtgtccacctttggactctcccttgtgaggcttgacattaggcaagagtcagataggcatactgatgtccttgatgccatcactacatacctggggataggatcttaccgtgaatggactgaagaacgccgccaagaatggttactgtctgaacttaatggcaagcgccctctgtttggctcagaccttcccaagactgaggaaatttctgatgttctagacacattccatgttattgctgagcttccctctgacaattttggtgcgtatatcatttccatggcgacagctccgtcggacgtccttgctgttgaactcctccaacgtgagtgtcatgtgaaaacaccacttagagtagtcccgctgtttgagaagttggccgatcttgaggctgccccggctgcattggccagactgttctcaatagattggtacagacagaggatcaatggcaagcaggaggtcatgattgggtattcagactcgggcaaagatgctggtcgcctctcagcagcttggcagctatacaaagctcaggaggagctcatcaaggttgctaaggactttggtgtgaagttaacgatgttccatggacgtggtgggactgttggaaggggtggtggtcccactcaccttgccatcttgtcccagccaccagacacgatccacggatcactcagggtcactgtgcaaggtgaagtcattgaacagtcatttggtgaggagcacttgtgctttaggacactgcaacgtttcacggctgctaccctggagcatggcatgcacccaccaaatgcaccaaagccagaatggcgagcccttcttgatgagatggcagttgtggcaactgaggaatatcggtccattgtcttcaaagagccacgctttgtcgagtatttccgccttgcaacccctgaaacagagtatggtaggatgaacataggaagcaggccatccaagagaaagccgagcggaggtatcgactcactccgagcaatcccatggatctttgcttggacacaaacgcggttccacctcccggtctggctaggatttggagccgcattcaagaatgtcctccagaaggacatcaggaacctccacatgctccaggaaatgtacaatgagtggccatttttcagggtgactattgatctggtggagatggtgttcgccaagggtaatcctggcattgccgcactgtatgacaaactcctcgtttcagaggaactgcatccattgggtgagaagctgagggccaactatgaggaaacccagaagcttctacttcaggttgctgggcacagggatcttctggaaggtgacctctacctgaagcagcggctccgcctgcgtgatgcgtacatcaccaccctgaacgtctgccaggcctacaccctgaagcggatccgtgacccggactaccatgtcgcgctgcgcccccacctctccaaggagatcatggactcgaccaaggctgcggcggacgtggtgaagctgaaccctggcagcgaatacgcaccggggctggaggacaccctcatcctgaccatgaagggcatagcagccggcctccagaacaccggttaa

SEQ ID NO: 3 - DNA, 2883 bp, Zea mays
atgccggagcggcaccagtccatcgacgcgcagctgcggctgctggcccccgggaaggtctccgaggacgacaagctcgtcgagtacgacgccctcctcgtcgaccgcttcctcgacatcctccaggacctgcacggcccccacctgcgcgaattcgtgcaggagtgctacgagctctcggcggagtacgagaacgaccgggatgaggctcggcttggcgagctcgggagcaagctcaccagcctgccgccgggggactccattgtcgtcgccagctccttctcgcacatgctcaacctcgctaacctcgccgaggaagtgcagatcgcgcaccgtcgccggatcaagctaaagcggggagacttcgccgacgaggcctccgcgccgacggagtcggacatcgaggagacgctcaagcgcctcgtctctcagctcggcaagtcgcgggaggaggtcttcgacgcgctcaagaaccagaccgtcgacctcgttttcaccgcccaccctacacagtccgtgaggaggtctcttctccagaagcatggaaggatccgtaactgcctgaggcagctttatgccaaggacatcactgctgacgacaagcaggagcttgatgaggcacttcagagggagattcaagctgctttcagaactgatgaaatccgcagaacccctcccactcctcaagatgaaatgcgtgctggaatgagttacttccatgaaactatatggaagggtgtaccaaaattcttgcgccgtattgacaccgctctgaaaaatattgggatcaatgaacgtctcccttacaatgctcctctcattcagttctcttcctggatgggtggtgatcgtgatggaaatccaagagtaacaccagaggttacacgggatgtatgcttgttggcgagaatgatggctgctaacttgtacttctctcagattgaagatttaatgtttgagctgtctatgtggcgctgcagtgatgaacttcggatccgtgcagatgaactacatcgctcttcaagaaaagctgcaaagcactatatagaattctggaagcaagttccgccaaatgaaccatatcgtgtcatacttggtgatgtcagggataaattgtactacacacgtgaacgttctcgtcacctattgacatctggaatttctgagattctagaggaggcaacttttactaatgttgaacagtttctggaacctcttgagctctgttatagatcattatgtgcttgtggtgacaaacctatagctgatggaagtcttcttgatttcttgcgtcaagtatcaacttttgggcttgcccttgtgaagctcgacatcaggcaggaatctgatcgacacactgatgtccttgattcgataacgacacatcttggaattggctcctatgctgaatggtctgaggagaaacgccaggattggctgttgtctgaactgaggggcaaacgtccattgtttggttcagatcttcctcagactgaagagactgctgatgttttaggcacatttcatgtccttgcagagcttcctgcagattgctttggcgcgtatatcatctctatggcaactgccccatccgatgtgcttgcggtcgagcttttgcagcgtgagtgccatgtaaaacatccactgagagttgttccactttttgagaaacttgcagatcttgaagcagctccagcagctgtagcacggctcttttcaattgactggtacatggataggattaatggcaagcaggaggtcatgattggatattcagactctggcaaagatgccgggcgtctctctgcagcatggcaaatgtataaagcacaagaagagctcatcaaagtggcaaagcattatggagtcaagttgacaatgtttcatgggaggggtggaacagttggcagaggaggtggtccaactcatctggccatattatctcagccaccagacacgatacatggatcacttcgtgtaacagttcaaggcgaagttattgagcactcctttggagaggagctcttgtgcttcagaactttgcaacgctacactgcagctactcttgagcatggcatgcatcctccgatttcccctaagccagaatggcgtgctctgatggatgaaatggctgttgtagcaactaaagaatatcgatcaattgtcttccaagaaccacgcttcgttgaatacttcagatcggctacacctgagactgaatatggtaggatgaatattggcagccgtccatcaaagaggaagcctagtgggggaatagaatcactccgtgcaattccatggatctttgcttggacacaaacaaggttccatctgcctgtctggcttggatttggtgcagcgataaagcacatcatgcagaaggacatcaggaacatccatatactgagagaaatgtacaatgagtggccattcttcagggtcacccttgatttgcttgagatggttttcgcgaagggagacccgggaatcgcagctgtgtacgacaaattgctagtggctgatgatttgcaatccttcggcgagcaactgaggaagaactatgaggagacaaaagagctactccttcaggttgctggtcacaaggacgtccttgaaggtgatccttatctgaagcagcgtctgcggctgcgtgagtcctacatcaccactctgaacgtgtgccaggcctacaccctgaagcggatacgcgacccgagcttccaggtgagcccgcagccgcccctgtccaaggagttcaccgacgagagtcagccggcggagctggtgcagctgaaccagcagagcgagtacgctccaggcctggaggacaccctcatcctgaccatgaagggcatcgccgccggcatgcagaacaccggctag

SEQ ID NO: 4 - DNA, 4476 bp, Artificial
  shuffled variant
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtaactaaccactgccgccgcccatttcttcttcgaccggttgccgcctgcgcgcggcactgctcgtacgtctccccgccagtgcttactgtaatgcatgcatgcaggtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctcgccgaggaggtgcagatcgcgcgccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccatcgacctcgtcttcaccgcgcatcccacacagtccgcccgccgctcgctcctgcaaaaaaatgccgggtatatatttttcaatggcttgatcgatatgctactcacgttatatacccttaagtcttaaccattattattatttttgataaataaaaatgtcggtcttgtcgctgcaggatccggaattgtctgacccagctgaatgccaaggacatcactgacgacgacaagcaggagctcgatgaggctctgcagagagaggtacgtacatattacatttcacaccagggaatgcaagaactttatcaagagacattcattctttgatagagatagaatagaacacatgcacagtacacgtggactcatgagcttgcaagacatcgagcacgacacgtgtaagttagtgcgccagagaaatcttcaatttatatgtcaagtcaggtcaggttctcccattaaaacacatataaataaatattcattattatcaagctaaggtaataaacaaccaaacttttccactatttaaactgtctttgcaaactccaaagtagaaactaacctaatcaggaaagaactagactgcacatttatgttttaacaatgcaatgagagaactgctacatgtataacagaattatttatatgaggccgacttgacttaagattcaatgttgaagaccacttgatgaaaactacactgaattatttatatgctattctccagctgtgctcaaagcattttccttttacttaaaaaagatcattttgtacaaagatctcttactcatatagagccatttgagtagaacttcggtaccacagatgcattaatggtttagttgtaatcaagttgttgtactcatcattatattttcctaacaagtagggcatccagtttctccttgatgaggaatcaaacctagatagccttaactccacaccctcaattagctaggctatgctcaagttcctagtgttacaaatttcagacgagtcataatgtcatcactgagcactcggtaaagagcgtctctctcatggtgcatatatatgatgcagaccacctgagaagtttactgcttcaagccaccaaagtggtatttttgttgtttgggttgtttagttctaattccttttcttgggtgttcacagatccaagcagccttcagaaccgatgaaatcaggagggcacaacccaccccccaggacgaaatgcgctatgggatgagctacatccatgagactgtatggaagggcgtgcctaagttcttgcgccgtgtggatacagccctgaagaatatcggcatcaatgagcgccttccctacaatgtttctctcattcggttctcttcttggatgggtggtgaccgcgatggtacatttctgcctacccttttcaataaagtggcaggagctctctgtctttcagcttgagagaaaccttcctgctttactctgactgcaatagatgttcagaaaaactagtctatcatttcgagctctcaggagctagaattttaaaatagaaattatttagtacacctcactaataaaaatttatcatccatacatgctagcacaacatataagcataatttaatcaaatctttatattgcaacctggaacctaacttgttgaattttttatatcacagaattatacgtgtagtattattttatatatcaaagagtgcttatattatatcagtacttgtcctgtcaatattcaaggctaacgtttttcttttctcgccagaaaattatatatacagaattatatgttttttctaagcctgtatatctttgcaatctatcgctatataggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccagagttcggctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaagggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgcattcaagtgggccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccatacctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 5 - DNA, 4476 bp, Artificial
  shuffled variant
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtaactaaccactgccgccgcccatttcttcttcgaccggttgccgcctgcgcgcggcactgctcgtacgtctccccgccagtgcttactgtaatgcatgcatgcaggtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctcgccgaggaggtgcagatcgcgcgccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccatcgacctcgtcttcaccgcgcatcccacacagtccgcccgccgctcgctcctgcaaaaaaatgccgggtatatatttttcaatggcttgatcgatatgctactcacgttatatacccttaagtcttaaccattattattatttttgataaataaaaatgtcggtcttgtcgctgcaggatccggaattgtctgacccagctgaatgccaaggacatcactgacgacgacaagcaggagctcgatgaggctctgcagagagaggtacgtacatattacatttcacaccagggaatgcaagaactttatcaagagacattcattctttgatagagatagaatagaacacatgcacagtacacgtggactcatgagcttgcaagacatcgagcacgacacgtgtaagttagtgcgccagagaaatcttcaatttatatgtcaagtcaggtcaggttctcccattaaaacacatataaataaatattcattattatcaagctaaggtaataaacaaccaaacttttccactatttaaactgtctttgcaaactccaaagtagaaactaacctaatcaggaaagaactagactgcacatttatgttttaacaatgcaatgagagaactgctacatgtataacagaattatttatatgaggccgacttgacttaagattcaatgttgaagaccacttgatgaaaactacactgaattatttatatgctattctccagctgtgctcaaagcattttccttttacttaaaaaagatcattttgtacaaagatctcttactcatatagagccatttgagtagaacttcggtaccacagatgcattaatggtttagttgtaatcaagttgttgtactcatcattatattttcctaacaagtagggcatccagtttctccttgatgaggaatcaaacctagatagccttaactccacaccctcaattagctaggctatgctcaagttcctagtgttacaaatttcagacgagtcataatgtcatcactgagcactcggtaaagagcgtctctctcatggtgcatatatatgatgcagaccacctgagaagtttactgcttcaagccaccaaagtggtatttttgttgtttgggttgtttagttctaattccttttcttgggtgttcacagatccaagcagccttcagaaccgatgaaatcaggagggcacaacccaccccccaggacgaaatgcgctatgggatgagctacatccatgagactgtatggaagggcgtgcctaagttcttgcgccgtgtggatacagccctgaagaatatcggcatcaatgagcgccttccctacaatgtttctctcattcggttctcttcttggatgggtggtgaccgcgatggtacatttctgcctacccttttcaataaagtggcaggagctctctgtctttcagcttgagagaaaccttcctgctttactctgactgcaatagatgttcagaaaaactagtctatcatttcgagctctcaggagctagaattttaaaatagaaattatttagtacacctcactaataaaaatttatcatccatacatgctagcacaacatataagcataatttaatcaaatctttatattgcaacctggaacctaacttgttgaattttttatatcacagaattatacgtgtagtattattttatatatcaaagagtgcttatattatatcagtacttgtcctgtcaatattcaaggctaacgtttttcttttctcgccagaaaattatatatacagaattatatgttttttctaagcctgtatatctttgcaatctatcgctatataggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctacgaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacaaggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaggaacttccaggtcctcaaagagatgtacaacgagtggccattcttcagggtcaccctggacctgctggagatgattttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 6 - DNA, 5042 bp, Artificial
  shuffled variant
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtaactaaccactgccgccgcccatttcttcttcgaccggttgccgcctgcgcgcggcactgctcgtacgtctccccgccagtgcttactgtaatgcatgcatgcaggtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctcgccgaggaggtgcagatcgcgcgccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccatcgacctcgtcttcaccgcgcatcccacacagtccgcccgccgctcgctcctgcaaaaaaatgccgggtatatatttttcaatggcttgatcgatatgctactcacgttatatacccttaagtcttaaccattattattatttttgataaataaaaatgtcggtcttgtcgctgcaggatccggaattgtctgacccagctgaatgccaaggacatcactgacgacgacaagcaggagctggacgaagcgttgcagcgcgaggtacgtacatattacatttcacaccagggaatgcaagaactttatcaagagacattcattctttgatagagatagaatagaacacatgcacagtacacgtggactcatgagcttgcaagacatcgagcacgacacgtgtaagttagtgcgccagagaaatcttcaatttatatgtcaagtcaggtcaggttctcccattaaaacacatataaataaatattcattattatcaagctaaggtaataaacaaccaaacttttccactatttaaactgtctttgcaaactccaaagtagaaactaacctaatcaggaaagaactagactgcacatttatgttttaacaatgcaatgagagaactgctacatgtataacagaattatttatatgaggccgacttgacttaagattcaatgttgaagaccacttgatgaaaactacactgaattatttatatgctattctccagctgtgctcaaagcattttccttttacttaaaaaagatcattttgtacaaagatctcttactcatatagagccatttgagtagaacttcggtaccacagatgcattaatggtttagttgtaatcaagttgttgtactcatcattatattttcctaacaagtagggcatccagtttctccttgatgaggaatcaaacctagatagccttaactccacaccctcaattagctaggctatgctcaagttcctagtgttacaaatttcagacgagtcataatgtcatcactgagcactcggtaaagagcgtctctctcatggtgcatatatatgatgcagaccacctgagaagtttactgcttcaagccaccaaagtggtatttttgttgtttgggttgtttagttctaattccttttcttgggtgttcacagatccaagcagccttcagaaccgatgaaatcaggagggcacaacccaccccccaggacgaaatgcgctatgggatgagctacatccatgagactgtatggaagggcgtgcctaagttcttgcgccgtgtggatacagccctgaagaatatcggcatcaatgagcgccttccctacaatgtttctctcattcggttctcttcttggatgggtggtgaccgcgatggtacatttctgcctacccttttcaataaagtggcaggagctctctgtctttcagcttgagagaaaccttcctgctttactctgactgcaatagatgttcagaaaaactagtctatcatttcgagctctcaggagctagaattttaaaatagaaattatttagtacacctcactaataaaaatttatcatccatacatgctagcacaacatataagcataatttaatcaaatctttatattgcaacctggaacctaacttgttgaattttttatatcacagaattatacgtgtagtattattttatatatcaaagagtgcttatattatatcagtacttgtcctgtcaatattcaaggctaacgtttttcttttctcgccagaaaattatatatacagaattatatgttttttctaagcctgtatatctttgcaatctatcgctatataggaaatccaagagttaccccggaggtgacaagagatgtatgcttgctggccagaatgatggctgcaaacttgtacatcgatcagattgaagagctgatgtttgaggtactgtacatccatactgcagatttgtttgattgaatgctctatgatttttttgcttgccctgttttttgctgtctccggtccataccagaactctcatgcatgcatcgtctgatatatctgtagctctctatgtggcgctgcaacgatgagcttcgtgttcgtgccgaagagctccacagttcgtctggttccaaagttaccaagtattacataggtaaccacaaacagaagcatttatgtttgcttaatttttgcctgccgtacagccttttgcaaaagtctccactagtgttttcaaattaatttgagggctcttttggcatcttttctgaagtgtatttgctggcgcagaattctggaagcaaattcctccaaacgagccctaccgggtgatactaggccatgtaagggacaagctgtacaacacacgcgagcgtgctcgccatctgcttgcgtcaggcgtgagcgagatttcagcggaagcgtcatttaccagtatcgaagaggtaaatatcgtcatgtatatatattatatatattcatagtatgacatcagcactgcaactaacaaaaaaaaaatcactactgtcgtgcatgcatgcagttccttgagccacttgagctgtgctacaaatcactgtgtgactgcggcgacaaggccatcgcggacgggagcctcctggacctcctgcgccaggttttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaagggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcgtgcgccagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacaaggcgcaggaggagatggcgcaggtggccaagcgctacggcgtcaagctcaccttgttccacggccgcggaggcaccgtgggcaggggtggcgggcccacgcaccttgccatcctgtcccaaccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctccgtcgtcgtcaaggagccgcgcttcgtcgagtacttcagatcggtatgctgccattgcccattgctttgtgacgatcgaattcatccatgtcgatcgttcttttcattcattcgagcgtttgtgcgtcactcactatcaggctacaccggagaccgagtacgggaggatgaacatcggcagccggccagccaagaggaggcccggcggcggcatcacgaccctgcgcgccatcccctggatcttctcgtggacccagaccaggttccacctccccgtgtggctgggagtcggcgccgcattcaagttcgccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcagaagaactcaagccctttgggaagcagctcagggacaaatacgtggagacacagcagcttctcctccaggtacaaaaaccagcaactcactgcactgcacttcacttcacttcactgtatgaataaaagtctggtgtctggttcctgatcgatgactgactactccactttgtgcagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaaccccgcgagcgagtacccgcccggcctggaagacacgctcatcctcaccatgaagggcatcgccgccggcatgcagaacactggctag

SEQ ID NO: 7 - DNA, 2913 bp, Artificial
  shuffled variant
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggtgcagatcgcgcgccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccgggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccagagttcggctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaagggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgcattcaagtgggccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccatacctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 8 - DNA, 2913 bp, Artificial
  shuffled variant
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggcgcagatcgcgcaccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccgggatccgaaattgtctgacccagctgaatgccaaggacatcactgacggcgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctacgaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacaaggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaggaacttccaggtcctcaaagagatgtacaacgagtggccattcttcagggtcaccctggacctgctggagatgattttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 9 - DNA, 2913 bp, Artificial
  shuffled variant
atggcgtcgaccaaggctcccggccccggcgagaagcaccactccatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggtgcagatcgcgcgccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccgggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagttgtacaacacacgcgagcgtgctcgccatctgctggcatctggagtttctgaaatttcagcggaagcttcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaagggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcgtgcgccagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacaaggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 10 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIARRRRNSKLKKGGFADEGSATTESDIEETLKRLVSEVGKSPEEVFEALKNQTVDLVFTAHPTQSARRSLLQKNAGIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELQSSAGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELKGKRPLLPPDLPQTDEIADVIGAFHVLAELPPDSFGPYIISMATAPSDVLAVELLQRECGIKQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKWAIDKDVKNFQVLKEMYNEWPFFRVTLDLLEMVFAKGDPGIAGLYDELLVAEELKPFGKQLRDKYVETQQLLLQIAGHKDILEGDPYLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 11 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEAQIAHRRRNSKLKKGGFADEGSATTESDIEETLKRLVSEVGKSPEEVFEALKNQTVDLVFTAHPTQSARRSLLQKNAGIRNCLTQLNAKDITDGDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELRGKRPLLPPDLPQTDEIADVIGAFHVLAELPPDSFGPYIISMATAPSDVLAVELLQRECGIKQPLPVVPLFERLADLQSAPASVERLFSVDWYMDRIKGKQQVMVGYSDSGKDAGRLSAAWQLYKAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMIFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 12 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIARRRRNSKLKKGGFADEGSATTESDIEETLKRLVSEVGKSPEEVFEALKNQTVDLVFTAHPTQSARRSLLQKNAGIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAEASFTSIEEFLEPLELCYKSLCDCGDKAIADGSLLDLLRQVFTFGLSLVKLDIRQESERHTDVIDAITTHLGIGSYREWSEDKRQEWLLSELKGKRPLLPPDLPQTDEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYKAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVKNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 13 - DNA, 2736 bp, Artificial
  shuffled variant
atggcgagcaccaaagcgccgggcccgggcgaaaaacatcattctatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgcccggctcacggggctggcccccgccgacgccatcctcgtggcgagctccatcctgcacatgctcaacctcgccaacctggccgaggaggcgcagatcgcgcaccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccaggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgcgaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaggaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccatacctgaagcaggggctggtgctgcgcaacccctacatcaccaccctgaacgtgttccaggcctacacgctgaagcggata

SEQ ID NO: 14 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGARLTGLAPADAILVASSILHMLNLANLAEEAQIAHRRRNSKLKKGGFADEGSATTESDIEETLKRLVSEVGKSPEEVFEALKNQTVDLVFTAHPTQSARRSLLQKNARIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELRGKRPLLPPDLPQTDEIADVIGAFHVLAELPPDSFGPYIISMATAPSDVLAVELLQRECGIKQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMVFAKGDPGIAGLYDELLVAEELKPFGKQLRDKYVETQQLLLQIAGHKDILEGDPYLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 15 - DNA, 2913 bp, Artificial
  shuffled variant
atggcgagcaccaaagcgccgggcccgggcgaaaaacatcattctatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggcgcagatcgcgcaccgccgccgcaacagcaagctcaagaaaggtgggttcgccgacgagggctccgccaccaccgagtccgacatcgaggagacactcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccaggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaagggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcgtgcgccagccgctgcccgtggtgccgctgttcgagaggctggtcgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacaaggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 16 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEAQIAHRRRNSKLKKGGFADEGSATTESDIEETLKRLVSEVGKSPEEVFEALKNQTVDLVFTAHPTQSARRSLLQKNARIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELKGKRPLLPPDLPQTDEIADVIGAFHVLAELPPDSFGPYIISMATAPSDVLAVELLQRECGVRQPLPVVPLFERLVDLQSAPASVERLFSVDWYMDRIKGKQQVMVGYSDSGKDAGRLSAAWQLYKAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVKNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 17 - DNA, 2913 bp, Artificial
  shuffled variant
atggcgagcaccaaagcgccgggcccgggcgaaaaacatcattctatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggtgcagatcgcgcaccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccgggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaagggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcgtgcgccagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 18 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIAHRRRNSKLKKGGFADEGSATTESDIEETLKRLVSEVGKSPEEVFEALKNQTVDLVFTAHPTQSARRSLLQKNAGIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELKGKRPLLPPDLPQTDEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVKNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 19 - DNA, 2913 bp, Artificial
  shuffled variant
atggcgagcaccaaagcgccgggcccgggcgaaaaacatcattctatcgacgcgcagctctgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggcgcagatcgcgctccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaaggaccagaccgtcgacctcgtcttcaccgcgcatcctacgcagtccgcccgccgctcgctcctgcaaaaaaatgccaggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacgcttcggctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgtacctcggcatcgggtcgtaccgcgagtggtccgaggacaagaggcaggagtggctgctgtcggagctgaaaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcgggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 20 - protein, 970 aa, Artificial
  shuffled variant
MASTKAPGPGEKHHSIDAQLCQLVPGKVSEDDKLIEYDALLVDRFLNILQDLHGPSLREFVQECYEVSADYEGKGDTTKLGELGAKLTGLAPADAILVASSILHMLNLANLAEEAQIALRRRNSKLKKGGFADEGSATTESDIEETLKRLVSEVGKSPEEVFEALKDQTVDLVFTAHPTQSARRSLLQKNARIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHASAGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTYLGIGSYREWSEDKRQEWLLSELKGKRPLLPPDLPQTDEIADVIGAFHVLAELPPDSFGPYIISMATAPSDVLAVELLQRECGIKQPLPVVPLFERLADLQSAPASVERLFSVDWYMDRIKGKQQVMVGYSDSGKDAGRLSAAWQLYKAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKWAIDKDVKNFQVLKEMYNEWPFFRVTLDLLEMVFAKGDPGIAGLYDELLVAEELKPFGKQLRDKYVETQQLLLQIAGHKDILEGDPFLKRGLVLRNPYITTLNVFQAYTLKRIRDPNFKVTPQPPLSKEFADENKPAGLVKLNPASEYPPGLEDTLILTMKGIAAGMQNTG

SEQ ID NO: 21 - DNA, 2913 bp, Artificial
  shuffled variant
atggcgagcaccaaagcgccgggcccgggcgaaaaacatcattctatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggtgcagatcgcgcgccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccgggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcaatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaagcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaaaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcatcaagcagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacaaggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaggaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 22 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIARRRRNSKLKKGGFADEGSATTESDIEETLKRLVSEVGKSPEEVFEALKNQTVDLVFTAHPTQSARRSLLQKNAGIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYINQIEELMFELSMWRCNDELRVRAEELHSSSGSKVTKYYIEFWKQIPPNEPYRVILGHVRDKLYNTRERARHLLASGVSEISAEASFTSIEEFLEPLELCYKSLCDCGDKAIADGSLLDLLRQVFTFGLSLVKLDIRQESERHTDVIDAITTHLGIGSYREWSEDKRQEWLLSELKGKRPLLPPDLPQTDEIADVIGAFHVLAELPPDSFGPYIISMATAPSDVLAVELLQRECGIKQPLPVVPLFERLADLQSAPASVERLFSVDWYMDRIKGKQQVMVGYSDSGKDAGRLSAAWQLYKAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 23 - DNA, 2913 bp, Artificial
  shuffled variant
atggcgagcaccaaagcgccgggcccgggcgaaaaacatcattctatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaggctcacggggctggcccccgccgacgccatcctcgtggcgagctccatcctgcacatgctcaacctcgccaacctggccgaggaggcgcagatcgcgcaccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccgggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacgcttcggctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgaaaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcgtgcgccagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaagaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggctag

SEQ ID NO: 24 - protein, 970 aa, Artificial
  shuffled variant
MASTKAPGPGEKHHSIDAQLRQLVPGKVSEDDKLIEYDALLVDRFLNILQDLHGPSLREFVQECYEVSADYEGKGDTTKLGELGARLTGLAPADAILVASSILHMLNLANLAEEAQIAHRRRNSKLKKGGFADEGSATTESDIEETLKRLVSEVGKSPEEVFEALKNQTVDLVFTAHPTQSARRSLLQKNAGIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHASAGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELKGKRPLLPPDLPQTDEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVKNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 25 - DNA, 2910 bp, Zea mays
atggcgagcaccaaagcgccgggcccgggcgaaaaacatcattctatcgacgcgcagctccgtcagctggtcccaggcaaggtctccgaggacgacaagctcatcgagtacgatgcgctgctcgtcgaccgcttcctcaacatcctccaggacctccacgggcccagccttcgcgaatttgtccaggagtgctacgaggtctcagccgactacgagggcaaaggagacacgacgaagctgggcgagctcggcgccaagctcacggggctggcccccgccgacgccatcctcgtggcgagctccatcctgcacatgctcaacctcgccaacctggccgaggaggtgcagatcgcgcaccgccgccgcaacagcaagctcaagaaaggtgggttcgccgacgagggctccgccaccaccgagtccgacatcgaggagacgctcaagcgcctcgtgtccgaggtcggcaagtcccccgaggaggtgttcgaggcgctcaagaaccagaccgtcgacctcgtcttcaccgcgcatcctacgcagtccgcccgccgctcgctcctgcaaaaaaatgccaggatccgaaattgtctgacccagctgaatgccaaggacatcactgacgacgacaagcaggagctcgatgaggctctgcagagagagatccaagcagccttcagaaccgatgaaatcaggagggcacaacccaccccgcaggatgaaatgcgctatgggatgagctacatccatgagactgtatggaagggtgtgcctaagttcttgcgccgtgtggatacagccctgaagaatatcggcatcaatgagcgccttccctacaatgtttctctcattcggttctcttcttggatgggtggtgaccgcgatggaaatccaagagttaccccggaggtgacaagagatgtatgcttgctggccagaatgatggctgcaaacttgtacatcgatcagattgaagagctgatgtttgagctctctatgtggcgctgcaacgatgagcttcgtgttcgtgccgaagagctccacagttcgtctggttccaaagttaccaagtattacatagaattctggaagcaaattcctccaaacgagccctaccgggtgatactaggccatgtaagggacaagctgtacaacacacgcgagcgtgctcgccatctgctggcatctggagtttctgaaatttcagcggaatcgtcatttaccagtatcgaagagttccttgagccacttgagctgtgctacaaatcactgtgtgactgcggcgacaaggccatcgcggacgggagcctcctggacctcctgcgccaggtgttcacgttcgggctctccctggtgaagctggacatccggcaggagtcggagcggcacaccgacgtgatcgacgccatcaccacgcacctcggcatcgggtcgtaccgcgagtggtccgaggacaagaggcaggagtggctgctgtcggagctgcgaggcaagcgcccgctgctgcccccggaccttccccagaccgacgagatcgccgacgtcatcggcgcgttccacgtcctcgcggagctcccgcccgacagcttcggcccctacatcatctctatggcgacggccccctcggacgtgctcgccgtagagctcctgcagcgcgagtgcggcgtgcgccagccgctgcccgtggtgccgctgttcgagaggctggccgacctgcagtcggcgcccgcgtccgtggagcgcctcttctcggtggactggtacatggaccggatcaagggcaagcagcaggtcatggtcggctactccgactccggcaaggacgccggccgcctgtccgcggcgtggcagctgtacagggcgcaggaggagatggcgcaggtggccaagcgctacggcgtcaagctcaccttgttccacggccgcggaggcaccgtgggcaggggtggcgggcccacgcaccttgccatcctgtcccagccgccggacaccatcaacgggtccatccgtgtgacggtgcagggcgaggtcatcgagttctgcttcggggaggagcacctgtgcttccagactctgcagcgcttcacggccgccacgctggagcacggcatgcacccgccggtctctcccaagcccgagtggcgcaagctcatggacgagatggcggtcgtggccacggaggagtaccgctcggtcgtcgtcaaggagccgcgcttcgtcgagtacttcagatcggctacaccggagaccgagtacgggaggatgaacatcggcagccggccagccaagaggaggcccggcggcggcatcacgaccctgcgcgccatcccctggatcttctcgtggactcagaccaggttccaccttcccgtgtggctgggagtcggcgccgccttcaagttcgccatcgacaaggacgtcaggaacttccaggtcctcaaagagatgtacaacgagtggccattcttcagggtcaccctggacctgctggagatggttttcgccaagggagaccccggcattgccggcttgtatgacgagctgcttgtggcggaagaactcaagccctttgggaagcagctcagggacaaatacgtggagacacagcagcttctcctccagatcgctgggcacaaggatattcttgaaggcgatccattcctgaagcaggggctggtgctgcgcaacccctacatcaccaccctgaacgtgttccaggcctacacgctgaagcggataagggaccccaacttcaaggtgacgccccagccgccgctgtccaaggagttcgccgacgagaacaagcccgccggactggtcaagctgaacccggcgagcgagtacccgcccggcctggaagacacgctcatcctcaccatgaagggcatcgccgccggcatgcagaacactggc

SEQ ID NO: 26 - protein, 970 aa, Zea mays
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIAHRRRNSKLKKGGFADEGSATTESDIEETLKRLVSEVGKSPEEVFEALKNQTVDLVFTAHPTQSARRSLLQKNARIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELRGKRPLLPPDLPQTDEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEPRFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

SEQ ID NO: 27 - DNA, 1353 bp, Zea mays
tggccgatcagttctttacttagctcgatgtaatgcacaatgttgatagtatgtcgaggatctagcgatgtaatggtgttaggacacgtggttagctactaatataaatgtaaggtcattcgatggtttttctattttcaattacctagcattatctcatttctaattgtgataacaaatgcattagaccataattctgtaaatatgtacatttaagcacacagtctatattttaaaattcttctttttgtgtggatatcccaacccaaatccacctctctcttcaatccgtgcatgttcaccgctgccaagtgccaacaacacatcgcatcgtgcatatctttgttggcttgtgcacggtcggcgccaatggaggagacacctgtacggtgcccttggtagaacaacatccttatccctatatgtatggtgcccttcgtagaatgacaccccttatccctacaatagccatgtatgcataccaagaattaaatatactttttcttgaaccacaataatttattatagcggcacttcttgttcaggttgaacacttatttggaacaataaaatgccgagttcctaaccacaggttcacttttttttttccttatcctcctaggaaactaaattttaaaatcataaatttaatttaaatgttaatggaaacaaaaaattatctacaaagacgactcttagccacagccgcctcactgcaccctcaaccacatcctgcaaacagacaccctcgccacatccctccagattcttcactccgatgcagcctacttgctaacagacgccctctccacatcctgcaaagcattcctccaaattcttgcgatcccccgaatccagcattaactgctaagggacgccctctccacatcctgctacccaattagccaacggaataacacaagaaggcaggtgagcagtgacaaagcacgtcaacagcaccgagccaagccaaaaaggagcaaggaggagcaagcccaagccgcagccgcagctctccaggtccccttgcgattgccgccagcagtagcagacacccctctccacatcccctccggccgctaacagcagcaagccaagccaaaaaggagcctcagccgcagccggttccgttgcggttaccgccgatcacatgcccaaggccgcgcctttccgaacgccgagggccgcccgttcccgtgcacagccacacacacacccgcccgccaacgactccccatccctatttgaacccacccgcgcactgcattgatcaccaatcgcatcgcagcagcacgagcagcacgccgtgccgctccaaccatctcgcttccgtgcttagcttcccgccgcgcc

SEQ ID NO: 28 - DNA, 1404 bp, Sorghum bicolor
gctttttccgtgctcaactattaactagtactcattattacctaattttcacttgtgatgacaattaatgcatcgatccacaattcagtaaatactttcatttaagcatatgtatagtattatacatttccaattcttcttttttgtgtggagatccacgacgatgcaagttgctcctcccaacccaaatccacctctctcttaaatccgcatatcttcaccaccaccagctgctacacatcgtattgtccaaatctgtgtcggcttgacccagtgatgtgcgcgctagatttggcagcgcctgaatgctgtgcagccacctgtatggtgcccttggtagagtaacaacacccttatccctacggcagccatgtatgacccttatccctacggcagccatgtataccaatacctttctttgaaccacaaaattatagtccatatccttaaccacaagttcattttttgtttcccggtctcctaaggaaattaagttctgtttccacagtttacatggatataggacatctatgttcctaacattaacattactggataacaggcaccctctcctccacaccctgcaaagccttcctccagcgccatgcatcctccgttgctaacagacacctctctccacatcgcgtgcaagcaaacctccaaattctaccgatccccagaatccggccttgactgcaaacagacacccctctccccatcctgcaaacccatcagccaaccgaataacacaagaaggcaggtgagcagtgacaaagcacgttaacagcagcaaagccaagccaaaaacgatccaggagcaaggtgcgccgcagctctcccggtcccctttgcggttaccactagctaagaatgaagatggtactctaaatgcatacttgcgcggtttttctctagtctaacttaataaactaaataaacaatttctttcttatttttttaatttagttcgtttagttagactagagaagaacacgaggagttatttgaagcatcgtccccatccttaccactagctagcactagcagacacccctctccacgtcctgcaaacaggcaatattagccagcggaataacacaagcaggcaagtgcgcagtgacaaagtacgtccacagcagcgatcccagccaaaagcagcgtagccacagccgcgcgcacgtctcggctacccttaccgccgatcacatgcatgcctttccaatcccgttgcacacgccgaccacacactcgccaactccccatccctatttgaagccaccggccggcgccctgcattgatcaatcaactcgcagcagaggagcagcacgagcaacacgccgcgccggtccaaccatctccagcttcgttcgcgcttcccggcccactccccggccgccgccgcc

SEQ ID NO: 29 - protein, 970 aa, Zea mays
MASTKAPGPGEKHHSIDAQLRQLVPGKVSEDDKLIEYDALLVDRFLNILQDLHGPSLREFVQECYEVSADYEGKGDTTKLGELGAKLTGLAPADAILVASSILHMLNLANLAEEVQIAHRRRNSKLKKGGFADEGSATTESDIEETLKRLVSEVGKSPEEVFEALKNQTVDLVFTAHPTQSARRSLLQKNARIRNCLTQLNAKDITDDDKQELDEALQREIQAAFRTDEIRRAQPTPQDEMRYGMSYIHETVWKGVPKFLRRVDTALKNIGINERLPYNVSLIRFSSWMGGDRDGNPRVTPEVTRDVCLLARMMAANLYIDQIEELMFELSMWRCNDELRVRAEELHSSSGSKVTKYYIEFWKQIPPNEPYRVILGHVRDKLYNTRERARHLLASGVSEISAESSFTSIEEFLEPLELCYKSLCDCGDKAIADGSLLDLLRQVFTFGLSLVKLDIRQESERHTDVIDAITTHLGIGSYREWSEDKRQEWLLSELRGKRPLLPPDLPQTEEIADVIGAFHVLAELPPDSFGPYIISMATAPSDVLAVELLQRECGVRQPLPVVPLFERLADLQSAPASVERLFSVDWYMDRIKGKQQVMVGYSDSGKDAGRLSAAWQLYRAQEEMAQVAKRYGVKLTLFHGRGGTVGRGGGPTHLAILSQPPDTINGSIRVTVQGEVIEFCFGEEHLCFQTLQRFTAATLEHGMHPPVSPKPEWRKLMDEMAVVATEEYRSVVVKEARFVEYFRSATPETEYGRMNIGSRPAKRRPGGGITTLRAIPWIFSWTQTRFHLPVWLGVGAAFKFAIDKDVRNFQVLKEMYNEWPFFRVTLDLLEMVFAKGDPGIAGLYDELLVAEELKPFGKQLRDKYVETQQLLLQIAGHKDILEGDPFLKQGLVLRNPYITTLNVFQAYTLKRIRDPNFKVTPQPPLSKEFADENKPAGLVKLNPASEYPPGLEDTLILTMKGIAAGMQNTG

(19) United States
(12) Patent Application Publication
(10) Pub. No.: US 2014/0298544 A1
(43) Pub. Date: Oct. 2, 2014

(54) Engineered PEP carboxylase variants for improved plant productivity

(71) Applicant: PIONEER HI BRED INTERNATIONAL INC, JOHNSTON, IA (US)

(72) Inventors: Guofu Li, Johnston, IA (US); Lu Liu, Palo alto, CA (US); Keith Roesler, Urbandale, IA (US); Daniel L Siehl, Menlo Park, CA (US); Genhai Zhu, San Jose, CA (US)

(21) Appl. No.: 14/354,643

(22) PCT Filed: Oct. 26, 2012
(86) PCT No.: PCT/US12/62038

§ 371 Date: Apr. 28, 2014

Related U.S. Application Data
(60) Provisional application No. 61/552,736, filed on Oct. 28, 2011.

(51) Int. Cl. C12N 15/82

(52) U.S. Cl. CPC C12N 15/8273; C12N 15/8271
USPC 800/287; 800/278; 800/298

(57) ABSTRACT

Variant phosphoenolpyruvate carboxylase (PEPC) genes are described. The encoded PEPC variants contain amino acid substitutions and have altered kinetic and/or regulatory properties with respect to wild-type PEPC. A variant PEPC gene may be expressed in a plant to improve one or more traits such as CO₂ assimilation rate, water use efficiency, and yield.

### FIELD OF THE INVENTION

The present invention relates to the field of plant molecular biology and improved plant performance, more particularly to the regulation of genes for improved drought tolerance and yield.

### BACKGROUND OF THE INVENTION

Insufficient water for optimum growth and development of crop plants is a major obstacle to consistent or increased food production worldwide. Population growth, climate change, irrigation-induced soil salinity, and loss of productive agricultural land to development are among the factors contributing to a need for crop plants which can tolerate drought. Drought stress often results in reduced yield, particularly reduced grain yield.

Plants are restricted to their habitats and must adjust to the prevailing environmental conditions of their surroundings. To cope with abiotic stressors in their habitats, higher plants use a variety of adaptations and plasticity with respect to gene regulation, morphogenesis, and metabolism. Adaptation and defense strategies may involve the activation of genes encoding proteins important in the acclimation or defense towards different stressors including drought. Understanding and leveraging the mechanisms of abiotic stress tolerance will have a significant impact on crop productivity. Methods are needed to enhance drought stress tolerance and to maintain or increase yield under conditions of drought or other abiotic stress.

### SUMMARY OF THE INVENTION

Methods are provided for improving plant performance, particularly for increasing drought tolerance in plants. In certain embodiments, yield of plants under drought conditions may be increased, relative to a control, by manipulating the water use efficiency (WUE) of the plant. More particularly, certain embodiments comprise introducing into a plant cell a polynucleotide that encodes a modified phosphoenolpyruvate carboxylase (PEPC) polypeptide. In certain embodiments, the polynucleotide encoding a variant PEPC is operably linked to a promoter that drives expression in a plant. Also provided are transformed plants, plant tissues, plant cells, and seeds thereof.

The stomata are the openings through which plants both lose water and gain the CO₂ needed for photosynthesis. Decreasing stomatal conductance may reduce water loss, but may also reduce internal CO₂ concentrations to such an extent that CO₂ fixation is reduced. This is particularly problematic when it occurs under conditions of adequate water availability. A means of maintaining CO₂ fixation rate when stomatal conductance is low would result in greater water use efficiency, achieving better drought tolerance or drought avoidance in plants.

Water use efficiency (WUE) can be calculated by dividing values for CO₂ fixation rate by values for stomatal conductance to assess the amount of CO₂ fixed per amount of water lost through stomata. Alternatively, WUE can be calculated as the amount of yield or biomass produced per amount of water used.

In corn and other plants that use C4 photosynthesis, PEP carboxylase (PEPC) is the enzyme that initially fixes CO₂ (after CO₂ is hydrated to bicarbonate by carbonic anhydrase). A kinetically superior PEPC might maintain CO₂ fixation rates at lower internal CO₂/bicarbonate concentrations that are present when stomatal conductance is low. Additionally, a kinetically superior PEPC may allow the plant to reduce stomatal conductance to conserve water.

Certain embodiments of the invention provide PEP carboxylase variants with improved kinetic properties relative to the wild-type PEPC. These variants, comprising multiple amino acid changes with respect to the wild-type, would be unlikely to arise from standard plant breeding techniques.

The following embodiments are among those encompassed by the invention:
1. A method of increasing the CO₂ assimilation rate, water use efficiency, yield under well-watered conditions, and/or yield under drought conditions, of a plant, comprising transforming said plant with a polynucleotide encoding PEP carboxylase.
2. The method of embodiment 1 wherein the polynucleotide has been modified to encode a PEP carboxylase protein with one or more altered kinetic or regulatory properties.
3. The method of embodiment 2 wherein the altered kinetic property is reflected in increased affinity (reduced S_{0.5} value) for bicarbonate.
4. The method of embodiment 2 wherein the altered kinetic property is reflected in increased K_{cat}/S_{0.5} values for the bicarbonate or PEP substrate.
5. The method of embodiment 2 wherein the altered regulatory property is reflected in increased activation by glucose-6-phosphate.
6. The method of embodiment 2 wherein the altered regulatory property is reflected in increased activation by glycine.
7. The method of embodiment 2 wherein the altered regulatory property is reflected in increased Ki for malate.
8. The method of embodiment 1 wherein expression of an endogenous PEP carboxylase gene is reduced.
9. The method of embodiment 1 wherein the plant is Zea mays.
10. The method of embodiment 1 wherein said polynucleotide is operably linked to a tissue-preferred promoter.
11. The method of embodiment 1 wherein stomatal conductance is decreased.
12. The method of embodiment 1 wherein there is no decrease in CO₂ fixation by the plant.
13. The method of embodiment 2 wherein altered enzymatic activity results in maintenance of CO₂ fixation rates when internal CO₂ or bicarbonate concentration is reduced.
14. The method of embodiment 1 wherein the polynucleotide is selected from the group consisting of SEQ ID NOS: 4-9, 13, 15, 17, 19, 21 and 23.
15. The method of embodiment 1 wherein the polynucleotide encodes a polypeptide selected from the group consisting of SEQ ID NOS: 10-12, 14, 16, 18, 20, 22 and 24.
16. The method of embodiment 1 wherein the polynucleotide encodes a polypeptide which, when compared to the polypeptide of SEQ ID NO: 26, has one or more altered kinetic or regulatory properties and comprises one or more amino acid substitutions selected from the group consisting of: R21C, K86R, V115A, H119R, H119L, N167D, R192G, D208G, D321N, H347Q, S348A, S350A, S404A, H472Y, R495K, V555I, R556K, A569V, R619K, F800W, R807K, V832I, F886Y and Q889R.
17. The method of embodiment 1 wherein said polynucleotide is operably linked to a stress-induced promoter.
18. The method of embodiment 2 wherein said polynucleotide to be modified is isolated from Zea mays.
19. The method of embodiment 2 wherein said modified polynucleotide is selected from the group consisting of SEQ ID NOS: 4-9, 13, 15, 17, 19, 21 and 23.
20. The method of embodiment 2 wherein expression of an endogenous PEPC gene is reduced.
21. An isolated polynucleotide of SEQ ID NO: 4-9, 13, 15, 17, 19, 21 or 23.
22. An isolated polypeptide of SEQ ID NO: 10-12, 14, 16, 18, 20, 22 or 24.
23. An isolated polypeptide which, when compared to the polypeptide of SEQ ID NO: 26, has one or more altered kinetic or regulatory properties and comprises at least three amino acid substitutions selected from the group consisting of: R21C, K86R, V115A, H119R, H119L, N167D, R192G, D208G, D321N, H347Q, S348A, S350A, S404A, H472Y, R495K, V555I, R556K, A569V, R619K, F800W, R807K, V832I, F886Y and Q889R.
24. A plant comprising a polynucleotide of SEQ ID NO: 4-9, 13, 15, 17, 19, 21 or 23.
25. A plant comprising a polypeptide of SEQ ID NO: 10-12, 14, 16, 18, 20, 22 or 24.
26. A plant comprising a polypeptide which, when compared to the polypeptide of SEQ ID NO: 26, has one or more altered kinetic or regulatory properties and comprises one or more amino acid substitutions selected from the group consisting of: R21C, K86R, V115A, H119R, H119L, N167D, R192G, D208G, D321N, H347Q, S348A, S350A, S404A, H472Y, R495K, V555I, R556K, A569V, R619K, F800W, R807K, V832I, F886Y and Q889R.
27. A plant as described in any of embodiments 24-26 wherein expression of an endogenous PEPC gene is reduced.

### BRIEF DESCRIPTION OF THE SEQUENCES

| | SEQ ID NO: |
|---|---|
| ZmPEPC C4 form (X15238; NCBI GI No. 22562) | 1 |
| ZmPEPC C3 form (X61489; NCBI GI No. 429148) | 2 |
| ZmPEPC root form (AB012228; NCBI GI No. 3132309) | 3 |
| ZmPEPC MOD1 Genomic | 4 |
| ZmPEPC MOD2 Genomic | 5 |
| ZmPEPC MOD3 Genomic | 6 |
| ZmPEPC MOD1 without introns (3D37F3) | 7 |
| ZmPEPC MOD2 without introns (3F30F12) | 8 |
| ZmPEPC MOD3 without introns (3C2H4) | 9 |
| ZmPEPC MOD1 - aa (3D37F3) | 10 |
| ZmPEPC MOD2 - aa (3F30F12) | 11 |
| ZmPEPC MOD3 - aa (3C2H4) | 12 |
| ZmPEPC MOD - 2A9E9 | 13 |
| ZmPEPC MOD - aa - 2A9E9 | 14 |
| ZmPEPC MOD - 2B5D5 | 15 |
| ZmPEPC MOD - aa - 2B5D5 | 16 |
| ZmPEPC MOD - 2B9F12 | 17 |
| ZmPEPC MOD - aa - 2B9F12 | 18 |
| ZmPEPC MOD - 3D10G8 | 19 |
| ZmPEPC MOD - aa - 3D10G8 | 20 |
| ZmPEPC MOD - 3C9C9 | 21 |
| ZmPEPC MOD - aa - 3C9C9 | 22 |
| ZmPEPC MOD - 3D5D12 | 23 |
| ZmPEPC MOD - aa - 3D5D12 | 24 |
| ZmPEPC WT | 25 |
| ZmPEPC WT - aa | 26 |
| ZmPEPC1-2 promoter | 27 |
| SB-C4PEPC promoter | 28 |
| AJ536629 | 29 |

### DETAILED DESCRIPTION OF THE INVENTION

In maize, sorghum, sugarcane and other plants that use C4 photosynthesis, CO₂ is initially fixed in mesophyll cells and is subsequently released in bundle sheath cells where rubisco is located, thus increasing CO₂ concentrations around rubisco and suppressing rubisco oxygenation and photorespiration. Phosphoenolpyruvate carboxylase (PEPC) is the enzyme in C4 plants that initially fixes CO₂ in mesophyll cells, after CO₂ is hydrated to bicarbonate by carbonic anhydrase. The PEPC reaction uses bicarbonate and the 3-carbon metabolite PEP as substrates to produce the 4-carbon metabolite oxaloacetic acid (OAA) and inorganic phosphate. There are variations among C4 plants in how the fixed carbon is shuttled into mesophyll cells, but in maize the OAA is converted to malate by malate dehydrogenase in mesophyll cell chloroplasts, and the malate then moves to the bundle sheath cells where it is decarboxylated by NADP-malic enzyme in chloroplasts. The CO₂ thus released is available as a substrate for rubisco, while the pyruvate produced in the decarboxylation reaction moves back to mesophyll cell chloroplasts where it is converted to PEP by pyruvate-orthophosphate dikinase to again provide PEP for the PEPC reaction.

In both C3 and C4 plants, PEPC isoforms are present with non-photosynthetic roles, such as replenishing TCA cycle intermediates. Certain embodiments of the invention relate to the PEPC genes encoding isoforms which serve a role in C4 photosynthesis, including the C4 PEPC gene from maize (see, for example, Genbank Accession Number AJ536629; Yanagisawa, et al., (1988) FEBS Letters 229:107-110; also, SEQ ID NOS: 1, 25 and 26). For more detailed background information on the topics of C4 photosynthesis and PEPC, see, Buchanan, Gruissem and Jones, Biochemistry & Molecular Biology of Plants. Rockville, Md.: American Society of Plant Physiologists, 2000. Print.

The C4 PEPC is a highly regulated enzyme, with several metabolites such as glucose-6-phosphate activating PEPC, and other metabolites, such as malate, inhibiting PEPC (Doncaster and Leegood, (1987) Plant Physiol 84:82-87). The enzyme is also regulated by phosphorylation at the conserved serine corresponding to serine 15 of the maize enzyme or serine 8 of the sorghum enzyme (Jiao, et al., (1991) Plant Physiol 96:297-301). PEPC is phosphorylated in the light, which decreases inhibition by malate and allows greater PEPC activity, and it is dephosphorylated in the dark.

The PEPC reaction exerts strong flux control over photosynthetic CO₂ assimilation in C4 plants, especially when internal CO₂ concentrations are low. In leaves of the C4 plant Amaranthus edulis, flux control coefficients for the PEPC reaction for CO₂ assimilation rate were determined to be 0.26 at ambient internal CO₂ concentrations, and 0.68 at low internal CO₂ concentrations (Dever, et al., (1997) Aust J of Plant Physiol 24:469-476). Internal CO₂ concentrations decrease during drought stress as stomatal conductance is decreased to reduce transpirational water loss from the plant. Thus, increasing PEPC activity in C4 plants may be especially helpful in maintaining CO₂ fixation during drought conditions.

Certain embodiments of the present invention provide variants of maize C4 PEPC with altered kinetic and regulatory properties relative to the wild-type or endogenous enzyme. These variants with improved properties were identified by expressing, in E. coli, libraries of genes encoding PEPC variants containing multiple amino acid substitutions, followed by PEPC activity assays and PEPC protein quantitation. Because these PEPC variants contain simultaneous, multiple amino acid substitutions, they would be difficult to obtain by traditional plant breeding methods. The altered properties of a PEPC variant may result in greater increases in CO₂ fixation, water use efficiency or yield than are achievable with wild-type C4 PEPC genes when overexpressed in maize, sorghum, sugarcane or other C4 plants.

PEPC polypeptides with altered enzymatic activity were identified by expressing maize PEPC gene (Yanagisawa, et al., (1988) FEBS Letters 229:107-110) shuffled libraries in E. coli, followed by PEPC activity assays and PEPC protein quantitation. Three rounds of screening of shuffled libraries were done. Three variants obtained from the third round of screening were named PEPC-MOD1 (SEQ ID NOS: 4, 7, 10), PEPC-MOD2 (SEQ ID NOS: 5, 8, 11) and PEPC-MOD3 (SEQ ID NO: 6, 9, 12). MOD1 has 10 amino acid substitutions and a higher affinity (lower S_{0.5} value) for bicarbonate. MOD2 and MOD3 have 7 and 6 mutations, respectively, and have better Vmax. All three variants (MOD1, 2, 3) showed increased activation by glucose-6-phosphate and glycine and increased Ki for malate.

Modified expression which provides greater total PEPC production or activity may result in increased CO₂ fixation rate, reduced stomatal conductance, equivalent levels of CO₂ fixation under drought or increased water use efficiency, relative to a control. Greater total PEPC activity may result with or without reduced expression of the native PEPC.

For polynucleotides, a “variant” comprises a deletion and/or addition of one or more nucleotides at one or more sites within the native polynucleotide and/or a substitution of one or more nucleotides at one or more sites in the native polynucleotide. As used herein, a “native” or “wild-type” or “endogenous” polynucleotide or polypeptide comprises a naturally occurring nucleotide sequence or amino acid sequence, respectively. For polynucleotides, conservative variants include those sequences that, because of the degeneracy of the genetic code, encode an amino acid sequence unchanged from the native polypeptide. Variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the endogenous protein disclosed. Generally, variants of a particular polynucleotide will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein.

Variants of a particular reference polynucleotide disclosed can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides is evaluated by comparison of the percent sequence identity shared by the two polypeptides they encode, the percent sequence identity between the two encoded polypeptides is at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention may be biologically active; that is, they continue to possess the desired biological activity of the native protein. Such variants may result from, for example, genetic polymorphism or from human manipulation. Biologically active variants may have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the amino acid sequence for the native protein as determined by sequence alignment programs and parameters described elsewhere herein. A biologically active variant of a reference protein may differ from that protein by as few as 1-15 amino acid residues, as few as 1-10, such as 6-10, as few as 5, as few as 4, 3, 2, or even 1 amino acid residue.

In certain embodiments, disclosed proteins may be altered in various ways including amino acid substitutions, deletions, truncations and insertions. Methods for such manipulations are generally known in the art. For example, amino acid sequence variants and fragments can be prepared by mutations in the DNA. Methods for mutagenesis and polynucleotide alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. The deletions, insertions and substitutions of the protein sequences may not produce radical changes in the characteristics of the protein; however, they may affect certain properties of the encoded protein. When it is difficult to predict the exact effect of a substitution, deletion, or insertion in advance of making such modifications, one skilled in the art will appreciate that the effect will be evaluated by routine screening assays known to those of ordinary skill in the art which may include, without limitation, antibody and enzyme-linked immunosorbent assays (ELISA), high-performance liquid chromatography (HPLC), gas chromatography/mass spectrometry (MS), and liquid chromatography/tandem mass spectrometry methods.

The following terms are used to describe the sequence relationships between two or more polynucleotides or polypeptides: (a) “reference sequence”, (b) “comparison window”, (c) “sequence identity” and (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence. A reference sequence may be the “native” or “wild-type” or “endogenous” sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12, and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990), supra. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997), supra. When utilizing BLAST, Gapped BLAST, PSI-BLAST, the default parameters of the respective programs (e.g., BLASTN for nucleotide sequences, BLASTX for proteins) can be used. See, the website of the National Center for Biotechnology Information of the National Library of Medicine of the National Institutes of Health. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3 and the nwsgapdna.cmp scoring matrix; % identity and % similarity for an amino acid sequence using GAP Weight of 8 and Length Weight of 2 and the BLOSUM62 scoring matrix or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see, Henikoff and Henikoff, (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As described herein, a nucleotide sequence encoding a polypeptide, variant or fragment thereof as provided herein may be operably linked to a promoter that drives expression of the sequence in a plant. Any one of a variety of promoters can be used with a nucleotide sequence, depending on the desired timing and location of expression. In some cases, the promoter is a constitutive promoter, a tissue-preferred promoter, a chemically-inducible promoter, a stress-inducible promoter, a light-responsive promoter or a diurnally-regulated promoter. For example, constitutive promoters can be used to drive expression of a nucleotide sequence of interest. The most common promoters used for constitutive overexpression are derived from plant virus sources, such as the cauliflower mosaic virus (CaMV) 35S promoter (Odell, et al., (1985) Nature 313:810-812). The CaMV 35S promoter delivers high expression in virtually all regions of transgenic monocot and dicot plants. Constitutive promoters also can include, for example, the core promoter of the Rsyn7 promoter and other constitutive promoters disclosed in WO 1999/43838 and U.S. Pat. No. 6,072,050; rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611.

Transgene expression can be beneficially adjusted by using a promoter suitable for the plant's background and/or for the type of transgene. Where low level expression is desired, weak promoters can be used. It is recognized that weak constitutive, weak inducible, or weak tissue-preferred promoters can be used. Generally, by “weak promoter” is intended a promoter that drives expression of a coding sequence at a low level. By low level is intended at levels of about 1/1000 transcripts to about 1/100,000 transcripts to about 1/500,000 transcripts. An example of a weak constitutive promoter is the GOS2 promoter; see, U.S. Pat. No. 6,504,083.

In some embodiments, tissue-preferred or developmental-preferred promoters can be used to drive expression of the sequence of interest in a tissue-preferred or a developmentally-preferred manner. For example, tissue-preferred promoters such as leaf-preferred promoter or root-preferred promoters can be used.

Leaf-preferred promoters are known in the art. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

Root-preferred promoters are also known and can be selected from the many available from the literature or isolated de novo from various compatible species. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean). See also, Bogusz, et al., (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. Leach and Aoyagi, (1991) describe their analysis of the promoters of the highly expressed rolC and rolD root-inducing genes of Agrobacterium rhizogenes (see, Plant Science (Limerick) 79(1):69-76). Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see, EMBO J. 8(2):343-350). The TR1′ gene, fused to nptII (neomycin phosphotransferase II) showed similar characteristics. Additional root-preferred promoters include the VfENOD-GRP3 gene promoter (Kuster, et al., (1995) Plant Mol. Biol. 29(4):759-772) and rolB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179. Other root-preferred promoters include Zm-NAS2 promoter (U.S. Pat. No. 7,960,613), Zm-Cyclo1 promoter (U.S. Pat. No. 7,268,226), Zm-Metallothionein promoters (U.S. Pat. Nos. 6,774,282; 7,214,854 and 7,214,855 (also known as RootMET2)), Zm-MSY promoter (US Patent Application Publication Number 2009/0077691) or MsZRP promoter (U.S. Pat. No. 5,633,363).

Other promoters may be utilized to drive expression of a polynucleotide of interest, such as the promoter of the maize KZM2 gene (see, Buchsenschutz, et al., (2005) Planta 222:968-976 and NCBI Accession Number AY919830; see also U.S. Provisional Patent Application Ser. No. 61/712,301, filed Oct. 11, 2012, incorporated herein by reference) or a green-tissue-preferred promoter (US Patent Application Publication Number 2011/0209242).

Constructs may also include one or more of the CaMV35S enhancer, Odell, et al., (1988) Plant Mol. Biol. 10:263-272, the ADH1 INTRON1 (Callis, et al., (1987) Genes and Dev. 1: 1183-1200), the UBI1ZM INTRON (PHI) as an enhancer and PINII terminator. Additionally or alternatively, other regulatory elements, including other terminators, may be used.

In some embodiments, a sequence of interest may be operably linked to a stress-inducible promoter, to drive expression of the sequence of interest in a stress-regulated manner. A stress-inducible promoter can be, for example, a rab17 promoter (Vilardell, et al., (1991) Plant Molecular Biology 17(5):985-993; Busk, et al., (1997) Plant J 11(6):1285-1295) or rd29a promoter (Yamaguchi-Shinozaki and Shinozaki, (1993) Mol. Gen. Genet. 236:331-340; Yamaguchi-Shinozaki and Shinozaki, (1994) Plant Cell 6:251-264).

Light-inducible and/or diurnally-regulated promoters can be used to drive expression of a nucleotide sequence in a light-dependent manner. A light-responsive promoter can be, for example, a rbcS (ribulose-1,5-bisphosphate carboxylase) promoter which responds to light by inducing expression of an associated gene. In some cases, diurnally-regulated promoters can be used to drive expression of a nucleotide sequence in a manner regulated by light and/or the circadian clock. For example, a cab (chlorophyll a/b-binding) promoter can be used to produce diurnal oscillations in gene transcription. In some embodiments, a diurnally-regulated promoter can be a promoter region as disclosed in U.S. patent application Ser. No. 12/985,413, herein incorporated by reference. In some embodiments, a promoter can be used that drives expression of a nucleotide sequence in a diurnally-regulated manner but further with a temporal expression pattern opposite of that of the endogenous sequence of interest.

An intron sequence can be added to the 5′ untranslated region or the coding sequence or the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, New York (1994).

Parameters such as gene expression level, water use efficiency, level or activity of an encoded protein, yield, and others are typically presented with reference to a control cell or control plant. A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed. A control may comprise numerous individuals representing one or more of the categories above; for example, a collection of the non-transformed segregants of category “c” is often referred to as a bulk null.

In another aspect, the present invention also provides methods for maintaining or increasing yield of a seed crop plant exposed to drought stress, where the methods include increasing expression of a variant polynucleotide provided herein.

Nucleotide sequences encoding variant PEPC polypeptides and/or other polynucleotides can be introduced into a plant. The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, breeding methods, stable transformation methods, transient transformation methods, and virus-mediated methods. “Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell targeted for transformation. For example, different methods may be preferred for use in monocots or in dicots. Suitable methods of introducing polypeptides and polynucleotides into plant cells include microinjection (Crossway, et al., (1986) Biotechniques 4:320-334), electroporation (Riggs, et al., (1986) Proc. Natl. Acad. Sci. USA 83:5602-5606, Agrobacterium-mediated transformation (U.S. Pat. No. 5,563,055 and U.S. Pat. No. 5,981,840), direct gene transfer (Paszkowski, et al., (1984) EMBO J. 3:2717-2722), and ballistic particle acceleration (see, for example, U.S. Pat. No. 4,945,050; U.S. Pat. No. 5,879,918; U.S. Pat. Nos. 5,886,244 and 5,932,782; Tomes, et al., (1995) in Plant Cell, Tissue, and Organ Culture: Fundamental Methods, ed. Gamborg and Phillips, (Springer-Verlag, Berlin); McCabe, et al., (1988) Biotechnology 6:923-926) and Lec1 transformation (WO 00/28058). See also, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and, 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, N.Y.), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens); all of which are herein incorporated by reference.

In specific embodiments, polynucleotide sequences of the invention can be provided to a plant using any of a variety of transient transformation methods. Such transient transformation methods include, but are not limited to, the introduction of the protein or variant thereof directly into the plant, or the introduction of the variant transcript into the plant. Such methods include, for example, microinjection or particle bombardment. See, for example, Crossway, et al., (1986) Mol Gen. Genet. 202:179-185; Nomura, et al., (1986) Plant Sci. 44:53-58; Hepler, et al., (1994) Proc. Natl. Acad. Sci. 91: 2176-2180 and Hush, et al., (1994) The Journal of Cell Science 107:775-784, all of which are herein incorporated by reference.

As indicated in some embodiments, the methods provided herein may rely upon the use of Agrobacterium-mediated gene transfer to produce regenerable plant cells having a nucleotide sequence of interest. Agrobacterium-mediated gene transfer exploits the natural ability of Agrobacterium tumefaciens to transfer DNA into plant chromosomes. Agrobacterium is a plant pathogen that transfers a set of genes encoded in a region called T-DNA of the Ti plasmid into plant cells at wound sites. The typical result of gene transfer by the native pathogen is a tumorous growth called a crown gall in which the T-DNA is stably integrated into a host chromosome. The ability to cause crown gall disease can be removed by deletion of the genes in the T-DNA without loss of DNA transfer and integration. The DNA to be transferred is attached to border sequences that define the end points of an integrated T-DNA.

A variety of Agrobacterium species are known in the art, particularly for monocotyledon transformation. Such Agrobacterium can be used in the methods of the invention. See, for example, Hooykaas, (1989) Plant Mol. Biol. 13:327; Smith, et al., (1995) Crop Science 35:301; Chilton, (1993) Proc. Natl. Acad. Sci. USA 90:3119; Mollony, et al., (1993) Monograph Theor Appl Genet 19:148; and Ishida, et al., (1996) Nature Biotechnol. 14:745; Komari, et al., (1996) The Plant Journal 10:165, herein incorporated by reference. See] also, DNA Cloning Service on the world wide web at DNA-cloning.com.

The Agrobacterium strain utilized in the methods of the invention can be modified to contain a gene or genes of interest, or a nucleic acid to be expressed in the transformed cells. The nucleic acid to be transferred is incorporated into the T-region and is flanked by T-DNA border sequences. In the Ti plasmid, the T-region is distinct from the vir region whose functions are responsible for transfer and integration. Binary vector systems have been developed where the manipulated disarmed T-DNA carrying foreign DNA and the vir functions are present on separate plasmids. In this manner, a modified T-DNA region comprising foreign DNA (the nucleic acid to be transferred) is constructed in a small plasmid which replicates in E. coli. This plasmid is transferred conjugatively in a tri-parental mating into A. tumefaciens which contains a compatible plasmid-carrying virulence gene. The vir functions are supplied in trans to transfer the T-DNA into the plant genome. Such binary vectors are useful in the practice of the present invention.

A vector comprising the nucleic acid of interest is introduced into an Agrobacterium. The term “introduced” is intended to mean providing a nucleic acid (e.g., expression construct) or protein into a cell (e.g., Agrobacterium). “Introduced” includes reference to the incorporation of a nucleic acid into a eukaryotic or prokaryotic cell where the nucleic acid may be incorporated into the genome of the cell, and includes reference to the transient provision of a nucleic acid or protein to the cell. The term “introduced” includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon or transiently expressed (e.g., transfected mRNA). General molecular techniques used in the invention are provided, for example, by Sambrook, et al., (eds.) Molecular Cloning: A Laboratory Manual, 1989, Cold Spring Harbor Laboratory Press, Cold Spring Harbor, N.Y.

Methods are known in the art for the targeted insertion of a polynucleotide at a specific location in the plant genome. In one embodiment, the insertion of the polynucleotide at a desired genomic location is achieved using a site-specific recombination system. See, for example, WO 1999/25821, WO 1999/25854, WO 1999/25840, WO 1999/25855 and WO 1999/25853, all of which are herein incorporated by reference. Briefly, the polynucleotide of the invention can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

In some cases, it is convenient to introduce nucleotide sequences of the invention as expression cassettes. Such expression cassettes can comprise 5′ and 3′ regulatory sequence operably linked to a disclosed polynucleotide. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein-coding regions, contiguous and in the same reading frame. The expression cassette may additionally contain at least one additional gene to be cotransformed into the organism. Alternatively, additional gene(s) can be provided on multiple expression cassettes. Expression cassettes can be provided with a plurality of restriction sites for insertion of the gene of interest to be under the transcriptional regulation of the regulatory regions. The expression cassette may additionally contain selectable marker sequences.

In some embodiments, an expression cassette will include in the 5′-3′ direction of transcription, a transcriptional and translational initiation region (i.e., a promoter), a polynucleotide disclosed herein and a transcriptional and translational termination region (i.e., termination region) functional in plants. The regulatory regions (i.e., promoters, transcriptional regulatory regions and translational termination regions) and/or the polynucleotide of the invention may be native/analogous to the host cell or to each other. Alternatively, the regulatory regions and/or the polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus or the promoter is not the native promoter for the operably linked polynucleotide.

While it may be optimal to express an exogenous sequence using a heterologous promoter, the native promoter sequence may be used. Such constructs can change expression levels in the plant or plant cell. Thus, the phenotype of the plant or plant cell can be altered.

The termination region may be native with the transcriptional initiation region, may be native with the operably linked polynucleotide of interest, may be native with the plant host, or may be derived from another source (i.e., foreign or heterologous) to the promoter, the polynucleotide of interest, the plant host or any combination thereof. Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gown, (1990) Plant Physiol. 92:1-11 for a discussion of host-preferred codon usage. Methods are available in the art for synthesizing plant-preferred genes. See, for example, U.S. Pat. Nos. 5,380,831 and 5,436,391 and Murray, et al., (1989) Nucleic Acids Res. 17:477-498, herein incorporated by reference. The plant preferred codons may be determined from the codons of highest frequency in the proteins expressed in a monocot or dicot of interest. Likewise, the optimized sequence can be constructed using monocot-preferred or dicot-preferred codons. See, for example, Murray, et al., (1989) Nucleic Acids Res. 17:477-498. It is recognized that all or any part of the gene sequence may be optimized or synthetic. That is, fully optimized or partially optimized sequences may also be used.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) (Virology 154:9-20), and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments; other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The variant polypeptides described herein may be used alone or in combination with additional polypeptides or agents to increase productivity of plants. For example, in the practice of certain embodiments, a plant can be genetically manipulated to produce more than one polypeptide associated with increased drought tolerance. Those of ordinary skill in the art realize that this can be accomplished in any of a number of ways. For example, each of the respective coding sequences for polypeptides described herein can be operably linked to a promoter and then joined together in a single continuous DNA fragment comprising a multigenic expression cassette. Such a multigenic expression cassette can be used to transform a plant to produce the desired outcome. Alternatively, separate plants can be transformed with expression cassettes containing one or a subset of the desired coding sequences. Transformed plants that exhibit the desired genotype and/or phenotype can be selected by standard methods available in the art such as, for example, immunoblotting using antibodies which bind to the proteins of interest, assaying for the products of a reporter gene, and the like. Then, all of the desired coding sequences can be brought together into a single plant through one or more rounds of cross-pollination utilizing the previously selected transformed plants as parents.

Methods for cross-pollinating plants are well known to those skilled in the art and are generally accomplished by allowing the pollen of one plant, the pollen donor, to pollinate a flower of a second plant, the pollen recipient, and then allowing the fertilized embryos in the pollinated flower to mature into seeds. Progeny containing the entire complement of desired coding sequences of the two parental plants can be selected from all of the progeny by standard methods available in the art for selecting transformed plants. If necessary, the selected progeny can be used as either the pollen donor or pollen recipient in a subsequent cross-pollination. Selfing of appropriate progeny can produce plants that are homozygous for added, heterologous genes. Back-crossing to a parental plant and out-crossing with a non-transgenic plant are also contemplated, as is vegetative propagation. Descriptions of other breeding methods that are commonly used for different traits and crop plants can be found in several references, e.g., Fehr, (1987), Breeding Methods for Cultivar Development, ed. J. Wilcox (American Society of Agronomy, Madison, Wis.).

The present invention may be used for transformation of any plant species, including, but not limited to, monocots and dicots. In some cases, plant species useful in the methods provided herein can be seed crop plants such as grain plants, oil-seed plants and leguminous plants. Of particular interest are plants where the seed is produced in high amounts, or the seed or a seed part is edible. Seeds of interest include the grain seeds such as wheat, barley, rice, corn (maize), rye, millet and sorghum. Plants of particular interest are corn, wheat and rice.

Examples of plant species of interest include, but are not limited to, corn (maize; Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus effiotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean and sugarcane plants are optimal, and in yet other embodiments corn plants are optimal.

Other plants of interest include grain plants that provide seeds of interest, oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

In certain embodiments the variant PEPC nucleic acid sequences can be used in combination (“stacked”) with other polynucleotide sequences of interest in order to create plants with a desired phenotype. The combinations generated can include multiple copies of any one or more of the polynucleotides of interest. The variant PEPC polynucleotides may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106 and WO 1998/20122) and high methionine proteins (Pedersen, et al., (1986) J. Biol. Chem. 261:6279; Kirihara, et al., (1988) Gene 71:359 and Musumura, et al., (1989) Plant Mol. Biol. 12:123)); increased digestibility (e.g., modified storage proteins (U.S. Pat. No. 6,858,778) and thioredoxins (U.S. Pat. No. 7,009,087)), the disclosures of which are herein incorporated by reference. The polynucleotides of the present disclosure can also be stacked with traits desirable for insect, disease or herbicide resistance (e.g., Bacillus thuringiensis toxic proteins (U.S. Pat. Nos. 5,366,892; 5,747,450; 5,737,514; 5,723,756; U.S. Pat. No. 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)) and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)) and polymers or bioplastics (e.g., U.S. Pat. No. 5,602,321; beta-ketothiolase, polyhydroxybutyrate synthase and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the variant PEPC polynucleotides with polynucleotides affecting agronomic traits such as male sterility (e.g., see, U.S. Pat. No. 5,583,210), stalk strength, flowering time or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes include, but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences; for example, antisense sequences of genes that negatively affect root development.

Additional, agronomic traits such as oil, starch and protein content can be genetically altered by transgenic and/or traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996 and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109), and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089), and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes. Other genes include kinases and those encoding compounds toxic to either male or female gametophytic development, or which encode a gene product which may interfere with male or female gametophyte function if appropriately targeted.

The quality of grain is reflected in traits such as levels and types of oils, saturated and unsaturated, quality and quantity of essential amino acids and levels of cellulose. In corn, modified hordothionin proteins are described in U.S. Pat. Nos. 5,703,049, 5,885,801, 5,885,802 and 5,990,389.

Commercial traits can also be encoded by a gene or genes that could increase for example, starch for ethanol production, or provide expression of proteins. Another commercial use of transformed plants is the production of polymers and bioplastics such as described in U.S. Pat. No. 5,602,321. Genes such as β-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

This disclosure can be better understood by reference to the following non-limiting examples. It will be appreciated by those skilled in the art that other embodiments may be practiced without departing from the spirit and the scope of the disclosure.

The articles “a” and “an” are used herein to refer to one or more than one (i.e., to at least one) of the grammatical object of the article. By way of example, “an element” may mean one element or more than one element.

### EXAMPLES

### Example 1

### Creation and Identification of PEPC Variants with Altered Properties

Libraries of modified PEPC polynucleotides were generated using recursive sequence recombination methods (Stemmer, Proc. Natl. Acad. Sci USA 91:10747-10751; Ness, et. al., Nature Biotechnology 20:1251-1255), also known as gene shuffling methods. These libraries incorporated diversity from related PEPC enzymes and also incorporated other designed and random changes. The starting polynucleotide sequence in which the diversity was incorporated was the maize C4 PEPC as shown in SEQ ID NO: 25. Each PEPC variant was expressed in E. coli as a thioredoxin fusion using the pET 32 expression vector (Novagen) with the E. coli expression strain Rosetta gami 2 BL21 DE3 pLYS S. Cultures expressing the variants were grown in 96-well culture blocks, lysed, and then PEPC activity and PEPC protein abundance were determined. The variants with high specific activity were subjected to further rounds of shuffling, for a total of 3 rounds. Examples of nucleotide and amino acid sequences of PEPC variants obtained with this method are SEQ ID NO: 4 to 24. Examples of amino acid substitutions in selected PEPC variants are summarized in Table 1.

TABLE 1
Amino acid substitutions in PEPC variants. The underlined amino acids are substitutions.

| PEPC | Alternate | Amino acid position | | | | | | | | | | |
|---|---|---|---|---|---|---|---|---|---|---|---|---|
| Variants | Name | Total | 21 | 86 | 115 | 119 | 167 | 192 | 208 | 321 | 347 | 348 |
| WT | | | R | K | V | H | N | R | D | D | H | S |
| 2A9E9 | | 5 | | R | A | | | | | | | |
| 2B5D5 | | 5 | | | A | | | | | | | |
| 2B9F12 | | 3 | | | | | | G | | | | |
| 3C9-C9 | | 8 | | | | R | | G | | N | | |
| 3D5-D12 | | 7 | | R | A | | | G | | | | A |
| 3D10-G8 | | 14 | C | | A | L | D | | | | | A |
| 3D37-F3 | MOD1 | 10 | | | | R | | G | | | Q | |
| 3F30-F12 | MOD2 | 7 | | | A | | | G | G | | | |
| 3C2-H4 | MOD3 | 6 | | | | R | | G | | | | |
| PEPC | Alternate | Amino acid position | | | | | | | | | | |
| Variants | Name | 350 | 404 | 472 | 495 | 555 | 556 | 569 | 619 | 800 | 807 | 832 |
| WT | | S | S | H | R | V | R | A | R | F | R | V |
| 2A9E9 | | | | | | I | K | | | | | |
| 2B5D5 | | | | | K | | | V | K | | K | |
| 2B9F12 | | | | | K | | | | | | K | |
| 3C9-C9 | | | A | | K | I | K | | K | | | |
| 3D5-D12 | | A | | | K | | | | | | K | |
| 3D10-G8 | | A | | Y | K | I | K | | K | W | K | |
| 3D37-F3 | MOD1 | A | | | K | I | K | | | W | K | |
| 3F30-F12 | MOD2 | | | | | I | K | | K | | | I |
| 3C2-H4 | MOD3 | | A | | K | | | | K | | K | |

| PEPC | Alternate | Amino acid position | |
|---|---|---|---|
| Variants | Name | 886 | 889 |
| WT | | F | Q |
| 2A9E9 | | Y | |
| 2B5D5 | | | |
| 2B9F12 | | | |
| 3C9-C9 | | | |
| 3D5-D12 | | | |
| 3D10-G8 | | | R |
| 3D37-F3 | MOD1 | Y | |
| 3F30-F12 | MOD2 | | |
| 3C2-H4 | MOD3 | | |

### Example 2

### Kinetic and Regulatory Properties of PEPC Variants

Detailed kinetic studies of PEPC variants were done in accordance with methods known in the art. See, for example, Tovar-Mendez et al. (2000) Plant Physiol. 123: 149-160; Tover-Mendez et al. (1998) Biochem. J. 332: 633-642.

Results are presented in Table 2. PEPC variants had several improved properties in comparison with wild type PEPC, including greater affinity (lower S_{0.5} values) with respect to the bicarbonate substrate, greater Kcat/S_{0.5} values with the bicarbonate or PEP substrates, greater activation (lower A_{0.5} values) with Glc-6-5 or glycine as activators and greater Ki values with malate as inhibitor.

TABLE 2
Kinetic and regulatory properties of PEPC variants.

| | HCO₃ | | | PEP, no activators | | PEP, with activators | | k_{cat}/S_{0.5} |
|---|---|---|---|---|---|---|---|---|
| | S_{0.5} | k_{cat}/S_{0.5} | k_{cat}/S_{0.5} | k_{cat}/S_{0.5} | k_{cat}/S_{0.5} | k_{cat}/S_{0.5} | k_{cat}/S_{0.5} | Fold |
| PEPC Variant | mM | sec⁻¹mM⁻¹ | % wt | sec⁻¹mM⁻¹ | % wt | sec⁻¹mM⁻¹ | % wt | incr. |
| WT | 0.0282 | 2122 | 100 | 34.1 | 100 | 2443 | 100 | 71.6 |
| 3D10-G8 | 0.0255 | 3200 | 151 | 86.2 | 253 | 4613 | 189 | 53.5 |
| 3C9-C9 | 0.021 | 3606 | 170 | 79.6 | 234 | 3831 | 157 | 48.1 |
| 3D5-D12 | 0.0277 | 3454 | 163 | 151 | 442 | 5551 | 227 | 36.9 |
| 3D37-F3 | 0.0167 | 3512 | 166 | 71.5 | 210 | 5010 | 205 | 70.1 |
| 3F30-F12 | 0.0248 | 3481 | 164 | 89.8 | 263 | 4496 | 184 | 50.1 |
| 3C2-H4 | 0.026 | 3581 | 169 | 64.1 | 188 | 3919 | 160 | 61.2 |
| | Glc-6-P | | | Glycine | | | | |
| PEPC | A_{0.5}, | A_{0.5}, | Vₘₐₓ | A_{0.5}, | A_{0.5}, | Vₘₐₓ | Malate | |
| Variant | mM | % wt | Fold incr | mM | % wt | Fold incr | Ki, mM | % wt |
| WT | 6.8 | 100 | 23.1 | 3.62 | 100 | 15.9 | 0.4 | 100 |
| 3D10-G8 | 0.92 | 14 | 2.5 | 0.23 | 6 | 3.6 | 5 | 1250 |
| 3C9-C9 | 3.85 | 57 | 11.4 | 0.76 | 21 | 11.5 | 2 | 500 |
| 3D5-D12 | 1.88 | 28 | 4.3 | 0.32 | 9 | 4.1 | 4 | 1000 |
| 3D37-F3 | 4.46 | 66 | 7.2 | 0.93 | 26 | 5.8 | 2.4 | 600 |
| 3F30-F12 | 5.11 | 75 | 12.6 | 0.7 | 19 | 11.3 | 3.3 | 825 |
| 3C2-H4 | 3.02 | 44 | 7.6 | 0.56 | 16 | 10.3 | 1 | 250 |

### Example 3

### Design of Constructs to Express PEPC Variants in Maize

Constructs were prepared to express the PEPC variants in maize. Expression was driven by PEPC promoters from maize (ZmPEPC1-2 pro) or sorghum (SbC4PEPC pro), but PEPC promoters from other plants, including C4 plants such as sugarcane, could also be used. In some constructs, introns were added to the cDNA of the PEPC variant to increase expression. When introns were added, the nucleotide sequence of the PEPC variant was referred to as “genomic” even if not all introns of the native gene were present. Examples of constructs expressing PEPC variants are Constructs A, B, C, D, and E, as shown:

| Construct Identifier | Promoter Source | Variant Name | Alternate Name |
|---|---|---|---|
| Construct A | Sorghum | 2B9F12 | |
| Construct B | Sorghum | 3C2H4 | |
| Construct C | Maize | 3F30F12 | MOD2 |
| Construct D | Maize | 3C2H4 | MOD3 |
| Construct E | Maize | 3D37F3 | MOD1 |

### Example 4

### Maize Transformation Methods

### A. Maize Particle-Mediated DNA Delivery

A DNA construct can be introduced into maize cells capable of growth on suitable maize culture medium. Such competent cells can be from maize suspension culture, callus culture on solid medium, freshly isolated immature embryos or meristem cells. Immature embryos of the Hi-II genotype can be used as the target cells. Ears are harvested at approximately 10 days post-pollination, and 1.2-1.5 mm immature embryos are isolated from the kernels, and placed scutellum-side down on maize culture medium.

The immature embryos are bombarded from 18-72 hours after being harvested from the ear. Between 6 and 18 hours prior to bombardment, the immature embryos are placed on medium with additional osmoticum (MS basal medium, Murashige and Skoog, (1962) Physiol. Plant 15:473-497, with 0.25 M sorbitol). The embryos on the high-osmotic medium are used as the bombardment target, and are left on this medium for an additional 18 hours after bombardment.

For particle bombardment, plasmid DNA is precipitated onto 1.8 mm tungsten particles using standard CaCl2-spermidine chemistry (see, for example, Klein, et al., (1987) Nature 327:70-73). Each plate is bombarded once at 600 PSI, using a DuPont Helium Gun (Lowe, et al., (1995) Bio/Technol 13:677-682). For typical media formulations used for maize immature embryo isolation, callus initiation, callus proliferation and regeneration of plants, see Armstrong, (1994) In “The Maize Handbook”, Freeling and Walbot, eds. Springer Verlag, NY, pp 663-671.

Within 1-7 days after particle bombardment, the embryos are moved onto N6-based culture medium containing 3 mg/I of the selective agent bialaphos. Embryos, and later callus, are transferred to fresh selection plates every 2 weeks. The calli developing from the immature embryos are screened for the desired phenotype. After 6-8 weeks, transformed calli are recovered.

### B. Transformation of Maize Using Agrobacterium

Agrobacterium-mediated transformation of maize is performed essentially as described by Zhao, et al., (2006) Meth. Mol. Biol. 318:315-323 (see also, Zhao, et al., (2001) Mol. Breed. 8:323-333 and U.S. Pat. No. 5,981,840 issued Nov. 9, 1999, incorporated herein by reference). The transformation process involves bacterium inoculation, co-cultivation, resting, selection and plant regeneration.

### 1. Immature Embryo Preparation:

Immature maize embryos are dissected from caryopses and placed in a 2 mL microtube containing 2 mL PHI-A medium.

### 2. Agrobacterium Infection and Co-Cultivation of Immature Embryos:

2.1 Infection Step:
PHI-A medium of (1) is removed with 1 mL micropipettor, and 1 mL of

Agrobacterium suspension is added. The tube is gently inverted to mix. The mixture is incubated for 5 min at room temperature.

2.2 Co-Culture Step:
The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

### 3. Selection of Putative Transgenic Events:

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

### 4. Regeneration of T0 Plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

### Media for Plant Transformation:

1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L Gelrite®, 100 μM acetosyringone (filter-sterilized), pH 5.8.
3. PHI-C: PHI-B without Gelrite® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4 D. After two weeks the tissue can be transferred to regeneration medium (Fromm, et al., (1990) Bio/Technology 8:833-839).

### Example 5

### Quantitation of PEPC RNA in Transgenic Plants Using qRT-PCR

Samples submitted for analysis were stored at −80 C until RNA isolation. RNA was isolated using the E.Z.N.A.® RNA kit (Omega Bio-Tek, Norcross, Calif., catalog #R1034-092) following manufacturer's conditions. The RNA was eluted in 60 μl of RNAse-free water and treated with 20 units of DNAse (Roche, Indianapolis, Ind.) following manufacturer's conditions. The DNAsed RNA was diluted with 4 volumes of 500 mM EDTA, pH 8 prior to inactivation of the DNAse by incubation at 65° C. for 30 minutes. The absence of DNA in the final RNA prep had been determined in a previous experiment for the same type and amount of tissue, using QRTPCR reactions (see below) containing Taq polymerase enzyme only (no reverse transcriptase enzyme). The purity and absence of inhibition by the RNA in QRTPCR reactions had been determined in a previous experiment for the same type and amount of tissue, using the Agilent BioAnalyzer (purity) and QRTPCR analysis of serially diluted RNA, which showed the expected dose-response (absence of inhibition). A normalization control assay was used to account for well-to-well RNA concentration differences and was designed to the sequence of the corn RNA polymerase II large subunit transcript. The normalization control transcript was found to have a constant relationship to the concentration of RNA in similar samples, in a separate experiment.

Real time QRTPCR assays were designed using Primer Express® 3.0 (Applied Biosystems, Foster, Calif.). All Tagman™ probes were quenched with the minor groove binder (MGB).

For analysis of the PEP carboxylase native and transgenic transcripts, two assays were designed. To detect the native PEPC, an assay was designed in the part of the native sequence that is not present in the transgenic construct. For analysis of the transgenic PEPC transcript, an assay to the 5-prime end of the UBQ3 terminator region was used. The PEPC and UBQ3 probes were both labeled with the fluorescent reporter dye FAM. The PEPC and normalization control assays were duplexed in the same reactions, and one replicate was analyzed.

Primers were obtained from Integrated DNA Technologies (Coralville, Iowa) and MGB probes were obtained from Applied Biosystems.

The one step QRTPCR was performed according to manufacturer's suggestions using the SuperScript® III Platinum® One Step QRTPCR kit (Invitrogen, Carlsbad, Calif., catalog #11745-500). Ten-microliter one-step QRTPCR reactions contained 5 microliters of 2× master mix, 0.2 μl of 50×SSIII/Platinum Taq/RNAse OUT mixture, 8 picomoles of each primer and 0.8 picomoles of each probe, 4 microliters of RNA and RNAse-free water to volume. The Applied Biosytems 7900 instrument was used for real time thermal cycling, with conditions of: 3 minutes at 50° C. (reverse transcription step), initial enzyme activation of 5 minutes at 95° C., and 40 cycles of 15 seconds at 95° C. and 1 minute at 60° C. (when fluorescence data is collected). Sequence Detection System version 2.2.1 was used for data collection and analysis. Calibrator samples were employed in all experiments in order to allow comparisons across experiments. The calibrator RNA sample for the ZM-PEPC native and transgenic assays was comprised of a pool of samples from constructs found to express both transcripts. A non-transgenic maize RNA sample was tested in all assays (B73).

The cycle threshold (Ct) data was exported from SDS software to Microsoft Excel. The delta delta Ct method was validated and employed for relative expression calculations (User Bulletin#2, Applied Biosystems). The relative expression of each gene of interest may be described as “fold expression of the gene of interest, relative to its expression in the calibrator, normalized to the expression of the corn RNA polymerase II LSU gene”.

The relative expression of the wild type and variant PEPC genes in T0 leaves of transgenic events for Construct B and Construct C are given in Table 3. This data confirmed that the RNA of the PEPC variants was present in leaves of transgenic plants.

TABLE 3
Quantitation of native and variant PEPC RNA in transgenic maize plants.

| | | Fold Relative |
|---|---|---|
| | Fold Relative Expression | Expression |
| | native | variant |
| | PEPC RNA | PEPC RNA |
| Construct B event ID | | |
| 120275678 | 26.76 | 7.73 |
| 120275686 | 22.15 | 9.17 |
| 120275694 | 23.16 | 6.65 |
| 120275682 | 19.40 | 4.78 |
| 120275695 | 17.65 | 6.00 |
| 120275683 | 19.17 | 7.69 |
| 120275699 | 19.37 | 8.13 |
| 120275679 | 18.30 | 5.24 |
| 120275685 | 20.06 | 5.98 |
| 120275677 | 20.30 | 7.08 |
| 120275697 | 19.10 | 37.76 |
| 120275702 | 18.27 | 4.49 |
| 120275684 | 16.17 | 4.62 |
| 120275676 | 16.75 | 5.30 |
| 120275700 | 21.87 | 7.27 |
| 120275688 | 16.83 | 4.66 |
| 120275681 | 21.72 | 8.94 |
| 120275693 | 26.45 | 15.24 |
| 120275698 | 22.23 | 4.31 |
| 120275696 | 20.40 | 10.06 |
| 120275689 | 19.13 | 7.40 |
| 120275680 | 19.30 | 4.59 |
| 120275687 | 19.93 | 5.38 |
| 120275692 | 17.55 | 5.45 |
| 120275701 | 19.27 | 0.01 |
| 120275691 | 22.29 | 0.00 |
| 120275690 | 19.72 | 5.85 |
| 120275707 | 20.75 | 5.63 |
| 120275704 | 15.62 | 6.53 |
| 120275705 | 18.83 | 0.79 |
| 120275703 | 22.19 | 17.56 |
| 120275706 | 22.65 | 6.86 |
| WT control | 20.11 | 0.00 |
| WT control | 27.12 | 0.00 |
| Construct C event ID | | |
| 124258208 | 43.12 | 7.72 |
| 124258207 | 47.14 | 28.93 |
| 124258145 | 43.94 | 0.07 |
| 124258149 | 45.37 | 25.73 |
| 124258148 | 43.83 | 28.88 |
| 124258144 | 43.06 | 28.42 |
| 124258147 | 39.38 | 27.35 |
| 124463539 | 31.98 | 16.20 |
| 124463637 | 41.21 | 24.29 |
| 124463530 | 44.66 | 35.20 |
| 124463640 | 37.15 | 18.77 |
| 124756721 | 48.60 | 30.01 |
| 124756720 | 40.34 | 27.99 |
| 124756722 | 47.67 | 35.98 |
| WT control | 35.27 | 0.09 |
| WT control | 40.69 | 0.05 |
| WT control | 41.60 | 0.05 |
| WT control | 35.74 | 0.07 |

### Example 6

### Quantitation of Variant PEPC Protein in Leaves Using Mass Spectrometry

Mass spectrometry was used to determine the quantities of variant PEPC protein in T0 leaves of transgenic maize plants, using the following materials and methods:

Materials. All chemicals were purchased from Sigma-Aldrich unless indicated otherwise. The extraction buffer T-CCLR contains 100 mM KP pH 7.8, 1 mM EDTA, 7 mM BME, 1% Triton, 10% Glycerol and 1× Protease Inhibitor (CalBiochem Cat#539137, Protease Inhibitor Cocktail Set V. EDTA-Free). The digestion buffer contains 50 mM ammonium bicarbonate (ABC) without adjusting pH.

Sample Preparation. A total of 500 iL of T-CCLR buffer was added per 10 leaf discs. Samples were mixed in a SPEX CertiPrep 2000 Geno/Grinder at a setting of 1600 strokes/min for 1 min, centrifuged briefly, repeated grinding once and then centrifuged (4° C., 3900 g) for 10 min. The supernatant collected was kept on ice, and total soluble proteins (TSPs) were measured with a Coomassie Protein Assay Reagent Kit (Pierce #23200). A total of 50 iL of supernatant was added to 110 iL of digestion buffer in polymerase chain reaction (PCR) tubes. An appropriate amount of recombinant protein was spiked to blank matrix and used as standard curve. An appropriate amount of sequencing grade modified trypsin (Promega) was added (trypsin/TSP ratio ˜1:15) to all samples including standard curve. Samples were mixed briefly and spun in a microcentrifuge. Samples were then placed in a homemade sample holder fitted into a CEM Discover® Proteomics System (Matthews, N.C.). Digestion lasted for 30 min (45° C., 50 W). After acidification with 10 iL of 10% (v/v) formic acid, samples were subject to LC-MS/MS analysis.

LC-MS/MS. The LC-MS/MS system included an AB SCIEX 4000 QTRAP® with a Turbo ion-spray source and Agilent 1100 LC. The autosampler temperature was kept at 6° C. during analysis. A total of 40 iL was injected onto an AQUASIL, 100×2.1 mm, 3 im, C18 column (ThermoFisher). LC was performed at a flow rate of 0.6 mL/min. Mobile phases consisted of 0.1% formic acid (MPA) and 0.1% formic acid in acetonitrile (MPB). The total run time for each injection was ˜28 min. Below is the detailed gradient table:

| Step | Total Time (min) | Flow Rate (μl/min) | A (%) | B (%) |
|---|---|---|---|---|
| 0 | 0.10 | 333 | 98.0 | 2.0 |
| 1 | 1.00 | 333 | 98.0 | 2.0 |
| 2 | 1.10 | 250 | 98.0 | 2.0 |
| 3 | 1.20 | 50 | 50.0 | 50.0 |
| 4 | 20.00 | 50 | 50.0 | 50.0 |
| 5 | 21.00 | 666 | 10.0 | 90.0 |
| 6 | 24.50 | 666 | 10.0 | 90.0 |
| 7 | 25.50 | 333 | 98.0 | 2.0 |
| 8 | 28.00 | 333 | 98.0 | 2.0 |

The mass spectrometer was operated in both multiple reaction monitoring (MRM) and linear ion-trap mode to select signature peptides. A complete list of MRM transitions was generated using MRM-initiated detection and sequencing (MIDAS) (AB Sciex) software for all tryptic peptides with an appropriate length (6-30 amino acids). The digested recombinant protein was analyzed using MRM-triggered information-dependent acquisition (IDA) to obtain both MRM chromatograms and MS/MS spectra, with the latter facilitating selection of the product ions with the highest sensitivity. The mass spectrometer was run in MRM mode at unit-mass resolution in both Q1 and Q3. The following electrospray ionization source parameters were used: dwell time, 200 ms for all MRM transitions; ion-spray voltage, 5500 V; ion source temperature, 555° C.; curtain gas (CUR), 20; both ion source gas 1 (GS1) and ion source gas 2 (GS2), 80; collision gas (CAD), high. The MRM transitions monitored:

For PEPC variant 3D37F3, also known as PEPC MOD1:

581.8/934.5, DILEGDPYLK (doubly charged) and 573.3/589.4, QEWLLSELK (doubly charged)

For PEPC variant 3F30F12, also known as PEPC MOD2:

696.9/738.4, 696.9/851.5, VTLDLLEMIFAK (doubly charged)

For PEPC variant 3C2H4, also known as PEPC MOD3:

540.3/879.5, LSAAWQLYK (doubly charged) and 573.3/589.4, QEWLLSELK (doubly charged)

Chromatograms were integrated using AB Sciex software Analyst® 1.4.2 with a Classic algorithm. Analyte peak areas were plotted against protein concentrations. A linear regression with 1/x² (where x=concentration) weighting was used for calibration curve fitting. The protein quantities of the PEPC variants in T0 leaves of transgenic events for Construct C, Construct B, Construct D, and Construct E are presented in Table 4. Events with values below the quantitation limit were given a value of 0. Most events have considerable amounts of a PEPC variant protein in T0 leaves. For Construct E, protein quantitation with two different peptides resulted in similar results. The PEPC variant proteins were not detected in wild type plants.

TABLE 4
Quantitation of variant PEPC protein in transgenic plants.

| | Variant PEPC protein |
|---|---|
| | (ppm) |
| Construct C | |
| event ID | |
| 124258208 | 211 |
| 124258207 | 10037 |
| 124258145 | 0 |
| 124258149 | 13762 |
| 124258148 | 14637 |
| 124258144 | 11606 |
| 124258147 | 13014 |
| 124463539 | 8468 |
| 124463637 | 13741 |
| 124463530 | 11223 |
| 124463640 | 9282 |
| 124756721 | 14502 |
| 124756720 | 7775 |
| 124756722 | 7890 |
| Construct B | |
| event ID | |
| 120275678 | 12454 |
| 120275686 | 5377 |
| 120275694 | 6309 |
| 120275682 | 4757 |
| 120275695 | 4660 |
| 120275683 | 4160 |
| 120275699 | 3021 |
| 120275679 | 7780 |
| 120275685 | 5318 |
| 120275677 | 6565 |
| 120275697 | 29463 |
| 120275702 | 9343 |
| 120275684 | 9881 |
| 120275676 | 10073 |
| 120275700 | 7696 |
| 120275688 | 8993 |
| 120275681 | 5023 |
| 120275693 | 10444 |
| 120275698 | 4266 |
| 120275696 | 11855 |
| 120275689 | 4062 |
| 120275680 | 8305 |
| 120275687 | 6278 |
| 120275692 | 6051 |
| 120275701 | 362 |
| 120275691 | 399 |
| 120275690 | 6172 |
| 120275707 | 5427 |
| 120275704 | 5634 |
| 120275705 | 224 |
| 120275703 | 10901 |
| 120275706 | 9683 |
| Construct D | |
| event ID | |
| 154573301 | 60319 |
| 154573314 | 207597 |
| 154573300 | 61986 |
| 154573293 | 58654 |
| 154573292 | 78057 |
| 154573297 | 58000 |
| 154573291 | 154101 |
| 154573298 | 74375 |
| 154573302 | 94480 |
| 154573304 | 0 |
| 154573306 | 0 |
| 154573307 | 72850 |
| 154573299 | 92388 |
| 154573290 | 87800 |
| 154573295 | 163845 |
| 154573294 | 64949 |
| 154573312 | 93323 |
| 154573305 | 58669 |
| 154573311 | 123678 |
| 154573296 | 101773 |
| 154573303 | 0 |
| 154573313 | 0 |
| 154573308 | 61381 |
| 154573316 | 5964 |
| 154573317 | 49094 |
| 154573319 | 61160 |
| 154573315 | 57251 |
| 154573309 | 125083 |
| 154573310 | 54380 |
| 154573318 | 61579 |

| | Variant PEPC | | | |
|---|---|---|---|---|
| | protein (ppm) | | | |
| Construct E | Using peptide | Using peptide | | standard |
| event ID | DILEGDPYLK | QEWLLSELK | Mean | deviation |
| 168676521 | 30320 | 33581 | 31950 | 2306 |
| 168676514 | 20078 | 23638 | 21858 | 2517 |
| 168676512 | 39583 | 39583 | 39583 | 0 |
| 168676515 | 35306 | 35652 | 35479 | 245 |
| 168676513 | 0 | 0 | 0 | 0 |
| 168676518 | 55787 | 58065 | 56926 | 1610 |
| 168676511 | 33903 | 33813 | 33858 | 64 |
| 168676520 | 0 | 0 | 0 | 0 |
| 168676522 | 6875 | 6635 | 6755 | 170 |
| 168676519 | 36769 | 33143 | 34956 | 2563 |
| 168676529 | 0 | 0 | 0 | 0 |
| 168676526 | 42483 | 40367 | 41425 | 1496 |
| 168676517 | 72400 | 73080 | 72740 | 481 |
| 168676528 | 39018 | 35318 | 37168 | 2616 |
| 168676516 | 0 | 0 | 0 | 0 |
| 168676525 | 0 | 0 | 0 | 0 |
| 168676535 | 42983 | 44131 | 43557 | 811 |
| 168676523 | 44609 | 47676 | 46142 | 2168 |
| 168676536 | 108446 | 103182 | 105814 | 3722 |
| 168676524 | 60080 | 49399 | 54740 | 7553 |
| 168676531 | 35539 | 38677 | 37108 | 2219 |
| 168676533 | 36054 | 39359 | 37707 | 2336 |
| 168676532 | 38640 | 39887 | 39263 | 881 |
| 168676530 | 23110 | 21316 | 22213 | 1269 |
| 168676534 | 35469 | 37556 | 36513 | 1475 |

### Example 7

### PEPC Activity in Leaves of Field Grown Transgenic Maize Plants

Field observation plots were grown at Johnston, Iowa for 9 transgenic events of Construct D in a maize hybrid and for 10 transgenic events of Construct E in a maize inbred. For each event, 5 leaf punches were obtained and combined (one each from 5 plants) from the upper fully expanded leaves at the V6 to V7 developmental stage. Bullet tubes were attached to the puncher so that samples went directly into the tubes, which were then frozen in liquid nitrogen, then transferred to a 96 well block in dry ice in the field and subsequently transferred to a −70° C. freezer. Protein extracts were prepared by grinding 3 times with 600 μl of extraction buffer in a GENO/GRINDER® 2000 for 30 sec each at 700 strokes per minute. Extraction buffer was 100 mM Hepes-KOH pH 7.3, 20% ethylene glycol, 1 mM MgCl₂, 0.5 mM EDTA, 3 mM DTT, 1 μl of Calbiochem® protease inhibitor cocktail III (catalog #539134) per 100 μl buffer, and 1 mM PMSF. Extracts were centrifuged for 2 min at 5000 g, and then supernatants were again centrifuged for 2 minutes at 20,000 g. Supernatants were frozen in liquid nitrogen and stored in a −70° C. freezer. Protein quantity was determined by a Bradford method, using the Coomassie PLUS™ reagent from Thermo Scientific (Product #23238) with bovine serum albumin standards. PEPC activity was determined at 25° C. with thawed extracts using 200 μl assay volumes in 96 well plates. The assay used the linking enzyme malate dehydrogenase to convert OAA product to malate, thereby converting NADH to NAD, which can be monitored easily by a decrease in absorbance at 340 nm. The final assay buffer was 50 mM Hepes-KOH, pH 7.3, 20% ethylene glycol, 5 mM MgCl₂, 10 mM NaHCO₃, 0.2 mM NADH, 3 units of MDH (Sigma M-1657), 2 μl of protein extract, and 4 mM PEP, with the PEP being added last to start the reaction, following 20 minutes at 25° C. without PEP to allow background to become negligible. Duplicate assays were done for each sample. Two control plots were sampled for each construct. The control for CONSTRUCT D was null seed of event ID154573291, and the control for CONSTRUCT E was wild type. PEPC specific activity values were expressed as μmol/min/mg protein, and are presented in Tables 5 and 6, ranked from highest activity events to lowest. Greater increases in PEPC activity were achieved with CONSTRUCT D than with CONSTRUCT E. For example, 8 of the 9 CONSTRUCT D events had PEPC activities at least 20% greater than the null mean, with the greatest increase being 77%. Only 5 of 10 CONSTRUCT E events had PEPC activities at least 20% greater than the WT mean, with the greatest increase being only 29%. These different increases in PEPC activity were achieved despite using an identical promoter for both constructs, and therefore may reflect differences in the properties of the two shuffled PEPC variants of these constructs.

TABLE 5
PEPC activity of CONSTRUCT D transgenic events.

| | | | | | | % |
|---|---|---|---|---|---|---|
| | CONSTRUCT | | | | | of |
| Event | D | PEPC Activity (umol/min/mg) | | | | Null |
| name | Event ID | Rep 1 | Rep 2 | Mean | SD | Mean |
| e1.6 | 154573291 | 1.27 | 1.38 | 1.33 | 0.08 | 177 |
| e1.8 | 154573292 | 1.23 | 1.23 | 1.23 | 0.00 | 164 |
| e3.9 | 154573301 | 1.27 | 1.01 | 1.14 | 0.18 | 152 |
| e5.5 | 154573309 | 1.19 | 1.08 | 1.13 | 0.07 | 152 |
| e2.2 | 154573293 | 0.96 | 0.99 | 0.98 | 0.02 | 131 |
| e3.5 | 154573298 | 0.92 | 1.00 | 0.96 | 0.06 | 128 |
| e3.2 | 154573297 | 0.92 | 0.92 | 0.92 | 0.00 | 123 |
| e3.7 | 154573300 | 0.89 | 0.92 | 0.90 | 0.02 | 121 |
| e3.11 | 154573302 | 0.81 | 0.82 | 0.82 | 0.01 | 109 |
| null | | 0.81 | 0.77 | 0.79 | 0.03 | |
| #1 | | | | | | |
| null | | 0.71 | 0.69 | 0.70 | 0.01 | |
| #2 | | | | | | |
| null | | 0.76 | 0.73 | 0.75 | 0.02 | |
| mean | | | | | | |

TABLE 6
PEPC activity of CONSTRUCT E transgenic events.

| | CONSTRUCT | | | | | % of |
|---|---|---|---|---|---|---|
| Event | E | PEPC Activity (umol/min/mg) | | | | WT |
| name | Event ID | Rep 1 | Rep2 | Mean | SD | Mean |
| e5.12 | 168676534 | 0.98 | 1.04 | 1.01 | 0.04 | 129 |
| e3.4 | 168676521 | 0.93 | 1.08 | 1.01 | 0.11 | 128 |
| e5.18 | 168676536 | 0.88 | 1.09 | 0.99 | 0.15 | 126 |
| e1.7 | 168676514 | 0.98 | 0.97 | 0.98 | 0.01 | 124 |
| e5.2 | 168676532 | 0.92 | 1.00 | 0.96 | 0.06 | 123 |
| e4.5 | 168676528 | 0.88 | 0.79 | 0.84 | 0.07 | 107 |
| e3.9 | 168676523 | 0.82 | 0.84 | 0.83 | 0.01 | 106 |
| e3.1 | 168676519 | 0.74 | 0.88 | 0.81 | 0.10 | 103 |
| e4.10 | 168676531 | 0.85 | 0.78 | 0.81 | 0.05 | 103 |
| e3.11 | 168676524 | 0.75 | 0.75 | 0.75 | 0.00 | 95 |
| WT #1 | | 0.82 | 0.69 | 0.76 | 0.10 | |
| WT #2 | | 0.77 | 0.86 | 0.82 | 0.07 | |
| WT | | 0.80 | 0.77 | 0.79 | 0.02 | |
| mean | | | | | | |

### Example 8

### Mature Plant and Ear Height of Field Grown Transgenic Maize Plants

Mature plant and ear heights were measured for CONSTRUCT D hybrid plants and CONSTRUCT E inbred plants grown with supplemental irrigation at Johnston, Iowa in an unusually dry summer. Measurements were done from the soil surface (avoiding brace roots) to the collar of the upper leaf for plant height, and to the ear node for ear height. Three field reps were measured, and the mean of multiple plant measurements was used for the rep values. For a rep, when available, 20 plants, 40 plants and 80 plants were measured, respectively, for transgenic events, for the null control for CONSTRUCT D and for the wild type (WT) control for CONSTRUCT E. The results are presented in Tables 7 through 10. Two tailed T-tests for statistical significance were done, and if a mean was significantly different from control at the p value of 0.05, it was marked with an asterisk. There were no significant differences in mature plant height for CONSTRUCT D or CONSTRUCT E events. For ear height, CONSTRUCT D event 3.9 and CONSTRUCT E event 5.2 were significantly reduced.

TABLE 7
Mature plant height of CONSTRUCT D transgenic events
CONSTRUCT D Plant Height (cm)

| Event | Rep1 | Rep2 | Rep3 | Mean | SE | vs. null |
|---|---|---|---|---|---|---|
| e3.11 | 259.9 | 259.7 | 252.6 | 257.4 | 2.42 | 5.7 |
| e1.8 | 255.2 | 248.6 | 257.5 | 253.7 | 2.68 | 2.1 |
| e3.2 | 250.7 | 255.9 | 254.5 | 253.7 | 1.54 | 2.0 |
| e2.2 | 257.6 | 253.7 | 245.9 | 252.4 | 3.45 | 0.7 |
| e3.7 | 249.8 | 252.4 | 253.0 | 251.7 | 0.98 | 0.0 |
| e3.5 | 251.6 | 248.7 | 250.9 | 250.4 | 0.86 | −1.3 |
| e3.9 | 251.6 | 247.9 | 247.4 | 249.0 | 1.34 | −2.7 |
| e5.5 | 245.1 | 251.1 | 250.4 | 248.9 | 1.91 | −2.8 |
| e1.6 | 247.8 | 241.8 | 248.3 | 246.0 | 2.11 | −5.7 |
| Null | 253.2 | 248.5 | 253.4 | 251.7 | 1.60 | |

TABLE 8
Mature ear height of CONSTRUCT D transgenic events.
CONSTRUCT D Ear Height (cm)

| | | | | | | vs. null |
|---|---|---|---|---|---|---|
| Event | Rep1 | Rep2 | Rep3 | Mean | SE | (cm) |
| e1.8 | 107.5 | 108.5 | 112.6 | 109.5 | 1.55 | 1.4 |
| e3.2 | 105.6 | 109.4 | 113.5 | 109.5 | 2.28 | 1.4 |
| e3.5 | 108.9 | 106.1 | 111.4 | 108.8 | 1.54 | 0.7 |
| e3.11 | 107.3 | 108.1 | 105.8 | 107.0 | 0.70 | −1.0 |
| e2.2 | 104.6 | 106.8 | 104.1 | 105.2 | 0.82 | −2.9 |
| e3.7 | 105.1 | 104.8 | 104.5 | 104.8 | 0.17 | −3.3 |
| e5.5 | 99.3 | 104.9 | 108.4 | 104.2 | 2.66 | −3.9 |
| e1.6 | 103.8 | 102.8 | 102.1 | 102.9 | 0.51 | −5.2 |
| e3.9 | 102.5 | 100.9 | 101.5 | 101.6* | 0.46 | −6.5 |
| Null | 104.6 | 109.0 | 110.7 | 108.1 | 1.83 | |

TABLE 9
Mature plant height of CONSTRUCT E transgenic events.
CONSTRUCT E Plant Height (cm)

| | | | | | | vs. WT |
|---|---|---|---|---|---|---|
| Event | Rep1 | Rep2 | Rep3 | Mean | SE | (cm) |
| e3.9 | 223.6 | 216.2 | 212.5 | 217.4 | 3.25 | 5.8 |
| e3.4 | 225.8 | 209.2 | 212.4 | 215.8 | 5.09 | 4.1 |
| e4.10 | 222.5 | 208.8 | 215.3 | 215.5 | 3.95 | 3.9 |
| e1.7 | 220.0 | 209.8 | 216.6 | 215.4 | 3.00 | 3.8 |
| e5.2 | 222.9 | 207.8 | 210.2 | 213.6 | 4.67 | 2.0 |
| e5.12 | 226.0 | 205.3 | 207.9 | 213.1 | 6.52 | 1.4 |
| e5.18 | 215.2 | 202.5 | 218.5 | 212.1 | 4.89 | 0.4 |
| e3.1 | 221.9 | 212.7 | 200.4 | 211.6 | 6.24 | 0.0 |
| e3.11 | 222.2 | 209.5 | 200.3 | 210.6 | 6.35 | −1.0 |
| e4.5 | 214.0 | 202.9 | 205.5 | 207.5 | 3.33 | −4.2 |
| WT | 217.5 | 209.3 | 208.2 | 211.6 | 2.95 | |

TABLE 10
Mature ear height of CONSTRUCT E transgenic events.
CONSTRUCT E Ear Height (cm)

| | | | | | | vs. WT |
|---|---|---|---|---|---|---|
| Event | Rep1 | Rep2 | Rep3 | Mean | SE | (cm) |
| e3.4 | 100.5 | 88.8 | 90.8 | 93.3 | 3.61 | 2.1 |
| e3.1 | 99.4 | 90.8 | 88.7 | 93.0 | 3.27 | 1.7 |
| e5.18 | 97.3 | 86.5 | 91.7 | 91.8 | 3.12 | 0.5 |
| e3.9 | 95.9 | 88.6 | 90.2 | 91.6 | 2.23 | 0.3 |
| e3.11 | 99.6 | 88.3 | 85.4 | 91.1 | 4.34 | −0.2 |
| e4.10 | 93.9 | 87.7 | 89.2 | 90.2 | 1.85 | −1.0 |
| e5.12 | 96.4 | 82.1 | 90.5 | 89.6 | 4.13 | −1.6 |
| e1.7 | 93.8 | 81.8 | 93.1 | 89.6 | 3.88 | −1.7 |
| e4.5 | 95.9 | 82.1 | 87.1 | 88.4 | 4.01 | −2.9 |
| e5.2 | 86.4 | 86.3 | 86.1 | 86.2* | 0.09 | −5.1 |
| WT | 95.7 | 89.5 | 88.6 | 91.3 | 2.20 | |

### Example 9

### Leaf Length, Leaf Width, and Leaf Area of Field Grown Transgenic Maize Plants

The length and width of mature ear leaves were measured for 3 transgenic events and for the wild type (WT) control for CONSTRUCT E inbred plants. Length was measured from collar to tip, and width was measured at the widest place. Area was then calculated as length×maximum width×0.75, using the method of Montgomery as cited by McKee, (1964) Agronomy J 56:240-241. Three field reps were measured at the irrigated Johnston location, and the mean of multiple plant measurements was used for the rep values. For a rep, when available, 20 plants and 60 plants were measured, respectively, for transgenic events and for the wild type (WT) control. Fewer plants were sometimes used if insufficient intact leaf tips were still present. The results are presented in Tables 11 through 13. Two tailed T-tests for statistical significance were done, and if a mean was significantly different from control at the p value of 0.05 or 0.01, it was marked with one or two asterisks, respectively. Two of the 3 transgenic events had significantly greater values for leaf area and for leaf length. The 3^{rd} transgenic event also had greater values for leaf area and length, although not statistically significant. Overexpression of the engineered PEPC variant of construct CONSTRUCT E resulted in approximately 4 to 6% greater leaf area compared with leaves of control plants.

TABLE 11
Ear leaf length of CONSTRUCT E transgenic events
Ear leaf length (cm)

| | | | | | | % of |
|---|---|---|---|---|---|---|
| Event | Rep1 | Rep2 | Rep3 | Mean | SE | WT |
| e5.18 | 85.3 | 83.8 | 85.7 | 84.9* | 0.56 | 104.1 |
| e4.5 | 84.9 | 84.2 | 85.2 | 84.8* | 0.31 | 103.9 |
| e3.9 | 86.3 | 83.5 | 83.2 | 84.3 | 0.99 | 103.4 |
| WT | 83.3 | 81.4 | 80.1 | 81.6 | 0.91 | 100.0 |

TABLE 12
Ear leaf width of CONSTRUCT E transgenic events
Ear leaf width (cm)

| | | | | | | % of |
|---|---|---|---|---|---|---|
| Event | Rep1 | Rep2 | Rep3 | Mean | SE | WT |
| e5.18 | 9.45 | 9.70 | 9.49 | 9.55 | 0.077 | 101.5 |
| e4.5 | 9.67 | 9.37 | 9.70 | 9.58 | 0.107 | 101.8 |
| e3.9 | 9.64 | 9.42 | 9.46 | 9.51 | 0.066 | 101.0 |
| WT | 9.40 | 9.36 | 9.48 | 9.41 | 0.034 | 100.0 |

TABLE 13
Ear leaf area of CONSTRUCT E transgenic events
Ear leaf area (cm²)

| | | | | | | % of |
|---|---|---|---|---|---|---|
| Event | Rep1 | Rep2 | Rep3 | Mean | SE | WT |
| e5.18 | 604.5 | 609.8 | 609.9 | 608.1** | 1.77 | 105.6 |
| e4.5 | 615.7 | 591.4 | 619.8 | 609.0* | 8.89 | 105.7 |
| e3.9 | 623.7 | 589.9 | 590.6 | 601.4 | 11.15 | 104.4 |
| WT | 586.5 | 571.5 | 569.9 | 576.0 | 5.30 | 100.0 |

### Example 10

### CO₂ Fixation Rate Stomatal Conductance and Water Use Efficiency of Transgenic Maize Plants

A LI-COR® LI-6400 portable photosynthesis system was used to measure CO₂ fixation rate and stomatal conductance to water on a leaf area basis in the field. Water use efficiency (WUE) was then calculated by dividing values for CO₂ fixation rate by values for stomatal conductance to determine the amount of CO₂ fixed per amount of water used. Observation plots of CONSTRUCT D hybrid plants were grown in Woodland, Calif. and subjected to severe stress at flowering time. Stable readings could not be obtained during the severe stress period. However, stable readings were obtained one day after rewatering by irrigation, and the results are presented in Tables 14 to 16. Three reps of 8 plants each were measured with a light intensity of 1500 μmol photons/m2/s, a reference chamber CO₂ concentration of 400 ppm, and ambient temperature. Event e3.9 had a statistically significant decrease of 17.3% in stomatal conductance. This event had a smaller decrease in CO₂ fixation rate, and thus had a significant increase of 10.5% in water use efficiency. Event e3.9 thus appeared to be using less water, and to be using it more efficiently. Event e1.6 also had decreased stomatal conductance and increased water use efficiency, but the changes were not statistically significant.

In contrast to the results with CONSTRUCT D, for CONSTRUCT E inbred plants there were no statistically significant differences in CO₂ fixation rate, stomatal conductance or water use efficiency observed in well watered (irrigated) conditions at Johnston, Iowa, as shown in Tables 17 to 19. However, the trend for slight increases in CO₂ fixation rate of 2.3%, 2.4%, and 3.5% for the 3 transgenic events, together with the statistically significant increases in leaf area for the same events at the same location, presented in Example 9, suggested that CO₂ fixation rate per plant may be increased for CONSTRUCT E events. For the CONSTRUCT E experiment, three reps of 4 plants each were measured with a light intensity of 2000 μmol photons/m2/s, a reference chamber CO₂ concentration of 400 ppm, and a constant block temperature of 28° C. The different LI-COR results between CONSTRUCT D and CONSTRUCT E could be due to differences in environmental conditions, differences in hybrids vs. inbreds, or differences in the properties of the different engineered PEPC variants.

TABLE 14
CO₂ fixation rate of CONSTRUCT D transgenic events.
CO₂ fixation rate (μmol CO₂/m2/s)

| | | | | | | % of |
|---|---|---|---|---|---|---|
| Event | Rep1 | Rep2 | Rep3 | Mean | SE | Null |
| Null | 36.7 | 36.4 | 37.2 | 36.8 | 0.237 | 100.0 |
| e1.6 | 35.0 | 33.6 | 38.2 | 35.6 | 1.357 | 96.9 |
| e3.9 | 33.4 | 32.1 | 35.2 | 33.6* | 0.882 | 91.4 |
| e2.2 | 31.7 | 37.4 | 33.3 | 34.1 | 1.700 | 92.8 |

TABLE 15
Stomatal conductance of CONSTRUCT D transgenic events
Stomatal conductance (mol H2O/m2/s)

| | | | | | | % of |
|---|---|---|---|---|---|---|
| Event | Rep1 | Rep2 | Rep3 | Mean | SE | Null |
| Null | 0.210 | 0.198 | 0.215 | 0.208 | 0.0051 | 100.0 |
| e1.6 | 0.188 | 0.171 | 0.205 | 0.188 | 0.0101 | 90.5 |
| e3.9 | 0.177 | 0.159 | 0.180 | 0.172* | 0.0064 | 82.7 |
| e2.2 | 0.177 | 0.209 | 0.195 | 0.194 | 0.0092 | 93.2 |

TABLE 16
Water use efficiency of CONSTRUCT D transgenic events
WUE (μmol CO₂/mol H2O)

| | | | | | | % of |
|---|---|---|---|---|---|---|
| Event | Rep1 | Rep2 | Rep3 | Mean | SE | Null |
| Null | 175 | 184 | 173 | 177 | 3.31 | 100.0 |
| e1.6 | 186 | 197 | 186 | 190 | 3.67 | 107.1 |
| e3.9 | 189 | 202 | 196 | 196* | 3.79 | 110.5 |
| e2.2 | 179 | 179 | 171 | 176 | 2.62 | 99.4 |

TABLE 17
CO₂ fixation rate of CONSTRUCT E transgenic events
CO₂ fixation rate (μmol CO₂/m2/s)

| | | | | | | % of |
|---|---|---|---|---|---|---|
| Event | Rep1 | Rep2 | Rep3 | Mean | SE | Null |
| WT | 45.0 | 40.5 | 43.0 | 42.9 | 1.30 | 100.0 |
| e4.5 | 45.8 | 44.4 | 41.3 | 43.8 | 1.32 | 102.3 |
| e5.18 | 43.4 | 42.8 | 45.4 | 43.9 | 0.76 | 102.4 |
| e3.9 | 43.0 | 44.6 | 45.4 | 44.4 | 0.70 | 103.5 |

TABLE 18
Stomatal conductance of CONSTRUCT E transgenic events
Stomatal conductance (mol H2O/m2/s)

| | | | | | | % of |
|---|---|---|---|---|---|---|
| Event | Rep1 | Rep2 | Rep3 | Mean | SE | Null |
| WT | 0.446 | 0.329 | 0.409 | 0.395 | 0.035 | 100.0 |
| e4.5 | 0.430 | 0.402 | 0.359 | 0.397 | 0.021 | 100.7 |
| e5.18 | 0.409 | 0.419 | 0.447 | 0.425 | 0.011 | 107.6 |
| e3.9 | 0.386 | 0.433 | 0.456 | 0.425 | 0.021 | 107.6 |

TABLE 19
Water use efficiency of CONSTRUCT E transgenic events
WUE (μmol CO₂/mol H2O)

| | | | | | | % of |
|---|---|---|---|---|---|---|
| Event | Rep1 | Rep2 | Rep3 | Mean | SE | Null |
| WT | 101.0 | 123.3 | 105.1 | 109.8 | 6.85 | 100.0 |
| e4.5 | 106.4 | 110.3 | 115.0 | 110.6 | 2.48 | 100.7 |
| e5.18 | 106.2 | 102.3 | 101.6 | 103.3 | 1.43 | 94.1 |
| e3.9 | 111.6 | 103.1 | 99.6 | 104.8 | 3.57 | 95.4 |

1. A method of increasing a plant's CO₂ assimilation rate, water use efficiency, yield under well-watered conditions, or yield under drought conditions, comprising transforming said plant with a polynucleotide encoding PEP carboxylase.

2. The method of claim 1 wherein the polynucleotide has been modified to encode a PEP carboxylase protein with one or more altered kinetic or regulatory properties.

3. The method of claim 2 wherein the altered kinetic property is reflected in increased affinity for bicarbonate.

4. The method of claim 2 wherein the altered kinetic property is reflected in increased K_{cat}/S_{0.5} values for the bicarbonate or PEP substrate.

5. The method of claim 2 wherein the altered regulatory property is reflected in increased activation by glucose-6-phosphate.

6. The method of claim 2 wherein the altered regulatory property is reflected in increased activation by glycine.

7. The method of claim 2 wherein the altered regulatory property is reflected in increased Ki for malate.

8. The method of claim 1 wherein expression of an endogenous PEP carboxylase gene is reduced.

9. The method of claim 1 wherein the plant is Zea mays.

10. The method of claim 1 wherein said polynucleotide is operably linked to a tissue-preferred promoter.

11. The method of claim 1 wherein stomatal conductance is decreased.

12. The method of claim 1 wherein there is no decrease in CO₂ fixation by the plant.

13. The method of claim 2 wherein CO₂ fixation rates are maintained, relative to a control, when internal CO₂ or bicarbonate concentration is reduced.

14. The method of claim 1 wherein the polynucleotide is selected from the group consisting of SEQ ID NO: 4-9, 13, 15, 17, 19, 21 and 23.

15. The method of claim 1 wherein the polynucleotide encodes a polypeptide selected from the group consisting of SEQ ID NOS: 10-12, 14, 16, 18, 20, 22 and 24.

16. The method of claim 1 wherein the polynucleotide encodes a polypeptide which, when compared to the polypeptide of SEQ ID NO: 26, has one or more altered kinetic or regulatory properties and comprises one or more amino acid substitutions selected from the group consisting of: R21C, K86R, V115A, H119R, H119L, N167D, R192G, D208G, D321N, H347Q, S348A, S350A, S404A, H472Y, R495K, V555I, R556K, A569V, R619K, F800W, R807K, V832I, F886Y and Q889R.

17. The method of claim 1 wherein said polynucleotide is operably linked to a stress-induced promoter.

18. The method of claim 2 wherein said polynucleotide to be modified is isolated from Zea mays.

19. The method of claim 2 wherein said modified polynucleotide is selected from the group consisting of SEQ ID NO: 4-9, 13, 15, 17, 19, 21 and 23.

20. The method of claim 2 wherein expression of an endogenous PEPC gene is reduced.

21. (canceled)

22. (canceled)

23. (canceled)

24. A plant comprising a heterologous polynucleotide of SEQ ID NO: 4-9, 13, 15, 17, 19, 21 or 23, wherein the plant exhibits an increased CO₂ assimilation rate, increased water use efficiency, increased yield under well-watered conditions, or increased yield under drought conditions, relative to a control.

25. (canceled)

26. A plant comprising a heterologous polypeptide which, when compared to the polypeptide of SEQ ID NO: 26, has one or more altered kinetic or regulatory properties and comprises one or more amino acid substitutions selected from the group consisting of: R21C, K86R, V115A, H119R, H119L, N167D, R192G, D208G, D321N, H347Q, S348A, S350A, S404A, H472Y, R495K, V555I, R556K, A569V, R619K, F800W, R807K, V832I, F886Y and Q889R.

27. A plant of claim 26 wherein expression of an endogenous PEPC gene is substantially reduced.

28. (canceled)
